(19) United States
(12) Patent Application Publication
(10) Pub. No.: US 2019/0255087 A1
(43) Pub. Date: Aug. 22, 2019

(54) ENCAPSULATION AND HIGH LOADING EFFICIENCY OF PHOSPHORYLATED DRUG AND IMAGING AGENTS IN NANOPARTICLES

(71) Applicant: THE PENN STATE RESEARCH FOUNDATION, University Park, PA (US)

(72) Inventors: James H. Adair, State College, PA (US); Gail L. Matters, Hummelstown, PA (US); Welley S. Loc, University Park, PA (US); Amra Tabakovic, State College, PA (US); Mark Kester, Harrisburg, PA (US); Sam Linton, Hershey, PA (US); Christopher McGovern, Harrisburg, PA (US); Xiaomeng Tang, University Park, PA (US); Gary A. Clawson, Bethesda, MD (US); Jill P. Smith, Washington, DC (US); Tye Deering, Charlottesville, VA (US)

(21) Appl. No.: 16/383,193

(22) Filed: Apr. 12, 2019

Related U.S. Application Data
(63) Continuation of application No. 15/411,377, filed on Jan. 20, 2017.
(60) Provisional application No. 62/281,970, filed on Jan. 22, 2016.

(51) Int. Cl. A61K 31/7072; A61K 31/513; C12N 15/115; A61K 9/51; A61K 31/7068

(52) U.S. Cl. CPC A61K 31/7072; A61K 31/513; C12N 15/115; C12N 2320/32; A61K 9/5115; A61K 31/7068; C12N 2310/16; A61K 9/5146

(57) ABSTRACT

Method of producing nanoparticle of drug and imaging agents are provided. The phosphorylated encapsulated drugs and imaging agents could be encapsulated at therapeutic levels, were encapsulated at higher amounts. The CPSNPs were more effective in treating cancer, in reducing cancer proliferation, arresting cancer cell growth than when not in the form of a CPSNP, and showed efficacious treatment of cancer cells at far lower dosage than free molecules. Calcium phosphosilicate and phosphate nanoparticles are disclosed and their method of use. The methods and nanoparticles are particularly efficacious where CPSNPs were used to encapsulate 5-FU metabolites such as FdUMP and gemcitabine metabolites.

### CROSS-REFERENCE TO RELATED APPLICATIONS

This application is a Continuation Application of U.S. Ser. No. 15/411,377 filed Jan. 20, 2017, which claims priority under 35 U.S.C. § 119 to Provisional Application U.S. Ser. No. 62/281,970, filed Jan. 22, 2016, all of which are incorporated herein by reference in their entirety.

This invention was made with support from the National Institutes of Health (NIH) under Grant R01CA167535 and R21CA170121 from the National Cancer Institute (NCI). This was also supported, in part, by the Grants UL1 TR000127 and TL1 TR000125 from the National Center for Advancing Translational Sciences (NCATS).

### REFERENCE TO GOVERNMENT SUPPORT

This invention was made with Government support under Grant Nos. CA167535 and CA170121 awarded by the National Institutes of Health/NCATS. The Government has certain rights in the invention.

### BACKGROUND OF THE INVENTION

Amorphous calcium phosphosilicate nanoparticles (CPSNPs) have been previously used to deliver a diverse range of therapeutic and imaging agents in biological systems.^{1, 2, 3 4, 5, 6, 7, 8, 9, 10, 11, 12, 13, 14, 15} Because CPSNPs are made of bioresorbable substances, calcium and phosphate, they are relatively non-toxic compared to heavy metal-based vehicles. CPSNPs are engineered to encapsulate chemotherapeutics or imaging agents within the particle matrix to enhance their biological half-life and pharmocokinetic properties during systemic delivery. Colloidal stability is maintained by covalently attaching polyethylene glycol (PEG) to the surface of CPSNPs, which prevents interactions with the reticuloendothelial system and allows circulation times for at least 96 hours in a murine animal model before clearance through the hepatobiliary duct and feces. The PEG also provides a platform for further bioconjugation of antibodies to target specific over expressed receptors on metastatic tumors.

The reverse-micelle synthetic scheme allows for explicit control over the final CPSNP diameter, ranging 15 to 200 nm. Particle diameters <200 nm can penetrate the cell membrane of drug resistant or highly fibrotic cancers such as pancreatic adenocarcinoma (PDAC). Controlled intracellular release of the drug is triggered by an acidic pH environment at late-stage endocytosis which causes the CPSNPs to dissolve into Ca²⁺(aq), HₓPO₄^{3-x}(aq) and Si(OH)₄⁰(aq).⁴ A change in the osmotic pressure from the dissolution ruptures the late endosome to release the bioresorbable products and active agents. Alternatively, external physiological fluids surrounding many types of solid tumors are typically low pH, which enables a high local release of the chemotherapeutics from CPSNPs within the vicinity. The ultimate goal is to deliver a therapeutic dose of active agents to the tumor of interest without inducing severe systemic side effects that is apparent with conventional chemotherapy.

Pancreatic adenocarcinoma (PDAC) patients often respond poorly to chemotherapeutic drugs, and even new drug combinations have demonstrated only a modest improvement in patient survival.¹⁶ This lack of efficacy has been attributed in part to poor drug delivery to tumor cells, since PDAC tumors are poorly vascularized with extensive desmoplastic stroma.¹⁷ Among the most common drugs used to treat PDAC patients are 5FU and gemcitabine, which act by blocking key enzymes in nucleotide synthesis.¹⁸ 5FU, a component of the FOLFIRINOX regiment, is metabolized to 5-fluoro-2′-deoxyuridine monophosphate (FdUMP) which, in the presence of 5,10-methylene-tetrahydrofolate (THF) as a methyl donor, irreversibly inhibits thymidylate synthase (TS).^{19,20} TS is a common target for chemotherapeutic drugs, since it can be inhibited both by folate analogs, such as raltitrexed, as well as nucleotide analogs such as 5FU/FdUMP. In addition, the efficacy of FdUMP can be enhanced by leucovorin, which increases the intracellular levels of CH₂THF and improves the binding of FdUMP to TS. TS inhibition results in nucleotide pool imbalances, misincorporation of nucleotides into DNA, inhibition of DNA synthesis as DNA polymerase stalls and replication forks collapse, and a reduction in DNA repair. 5FU-treated cells undergo cell cycle arrest and apoptosis.¹⁶

Similarly, the prodrug gemcitabine (2′,2′-difluorodeoxycytidine or dFdC) is intracellularly phosphorylated by deoxycytidine kinase (dCK) to form dFdCMP (also referred to as GemMP, dFdCDP and dFdCTP (62). Gemcitabine has multiple modes of action; gemcitabine diphosphate (dFdCDP) inhibits ribonucleotide reductase (RR), which is responsible for producing the deoxynucleotides required for DNA synthesis and repair. This favors dFdCTP incorporation into DNA, resulting in stalled DNA replication forks and apoptosis (62). Thus, both 5-FU and gemcitabine are activated within tumor cells by conversion to phosphorylated drug metabolites.

There are many drawbacks with 5FU that limit its use, including severe side effects from systemic administration (e.g. bone marrow suppression, cardiomyopathy, neurotoxicity), metabolic inactivation and rapid clearance. Each of these factors contributes to the high 5FU doses required for maximum therapeutic efficacy. It has been well documented that patient to patient differences also can influence both the efficacy of 5FU and the severity of drug side-effects. In vivo studies have shown that less than 20% of the 5FU pro-drug becomes activated to FdUMP; more than 80% of 5FU is catabolized to the inactive 5FU metabolite 5-fluorodihydrouracil by the enzyme dihydropyrimidine dehydrogenase (DPD).¹⁶ Colon cancer patients with a DPD mutation that causes a partial enzymatic inactivation (the IVS14+1G>A SNP, rs3918290, known as DPYD*2A) have 5FU clearance rates 2.5 times lower than wild-type controls and are at increased risk for 5FU induced toxicities.²¹ Many patients treated with 5FU have significant off-target effects due to the accumulation of toxic 5FU metabolites and increased dosing of 5FU directly correlates with cardiovascular and gastrointestinal toxicity, giving 5FU a narrow therapeutic index.²² Metabolites of 5FU and 5FU that differ only slightly in their composition can have vastly different side-effects, toxicities and efficacies.

Likewise, gemcitabine can be inactivated by cytidine deaminase (CDA) and rapidly cleared from the body (28-30). Both gemcitabine and 5-FU are transported into tumor cells by nucleoside transporter systems, including human equilibrative nucleoside transporters (hENTs), which have low affinity for FdUMP or dFdCMP (31).

Others have reported that FdUMP[10] and GemMP[10], a synthetic polymer of ten FdUMP and GemMP molecules, are highly active in preclinical models of acute myeloid leukemia, acute lymphoblastic leukemia, glioblastoma, thyroid, and prostate cancers. 27, 28, 29, 30, 31, 32, 33, 34, 35, 36, 37, 38, 39, 40, 41, 42, 43 FdUMP[10] and GemMP[10] effectively blocked the growth of multiple cancer cell lines in vitro and had anti-tumor activity in vivo. However, since the metabolic breakdown and side-effects associated with 5FU limit its utility and FdUMP and GemMP has been shown to be effective, direct delivery of FdUMP and GemMP to tumors with CPSNPs could further increase its efficacy and reduce off-target toxicities.

### SUMMARY

Methods of encapsulation and high loading efficiency of active phosphorylated drugs and imaging agents is provided. In an embodiment, prodrug chemotherapeutic agents, FdUMP and GemMP, in calcium phosphosilicate nanoparticles have increased efficiency and increased capacity to inactivate cancer cell proliferation, arrest cancer cells prior to G1 phase, inhibited thymidylate synthase compared to the non-phosphorylated counterparts, of the drug, in one embodiment, compared to 5FU, FUdR, and Gem. The presence of a phosphate group allows phosphorylated agents to adsorb and become encapsulated at therapeutic doses. These potent active drugs in CPSNPs is directly delivered to cells, which makes treatment highly efficacious at FdUMP doses up to ˜1000× less than the free drug in vitro on human pancreatic cancers. Methods of making such encapsulated drugs and/or imaging agents, methods of treating cancer cells, methods of treating cancer and compositions comprising same are described.

### BRIEF DESCRIPTION OF THE DRAWINGS

FIG. 1 is a graphic showing a schematic of the calcium phosphosilicate nanoparticle double reverse-micelle synthesis and encapsulation procedure. Micelle water pool size is maintained by the water-to-surfactant mole ratio, ω=[H2O]/[Igepal CO-520]=4. Microemulsions A and B consist the aqueous calcium chloride, and hydrogen phosphate/metasilicate/drug precursors, respectively. Immediately following micellar exchange from combining A and B, sub-particles form as phoshorylated agents (black dots) adsorb to, or get encapsulated by, the calcium phosphate material. Secondary nucleation and growth complete the nanoparticle and the reaction is quenched with sodium citrate. This Cit-X-CPSNP suspension is laundered with a van der Waals HPLC system equipped with a UV/Vis spectrophotometer to monitor the launder cycles. The laundered nanoparticles are PEGylated to obtain mPEG-X-CPSNPs. X=5FU, 5FU:ATP, FdUMP, FUdR, Gem, GemMP, or Ghost (negative CPSNP control). Micelles and CPSNPs are not drawn to scale.

FIG. 2 shows the structures of the encapsulated drug agents in this study, 5-fluorouracil (5FU), 5-fluorouracil, adenosine 5′-triphosphate (5FU:ATP), 5-fluoro-2′-deoxyuridine (FUdR), 5-fluoro-2′-deoxyuridine monophosphate (FdUMP), gemcitabine (Gem), and gemcitabine monophosphate (GemMP).

FIG. 3 are two graphs of therapeutic effect tested in vitro on BxPC-3 and PANC-1 cell lines after centrifugation and van der Waals HPLC laundering as described in Example 1.

FIGS. 4A-4C are a micrograph and graphs. FIG. 4A shows the lognormal particle size distribution and transmission electron microscopy micrograph with enlarged inset of a selected surface-modified mPEG-FdUMP-CPSNP formulation. The lognormal mean by diameter for mPEG-FdUMP-CPSNPs is 57±1 nm. FIG. 4B shows lognormal particle size distribution for mPEG-Ghost-CPSNPs. The lognormal mean by diameter for mPEG-Ghost-CPSNPs is 30±1 nm. FIG. 4C summarizes the surface characterization where the average zeta potentials of five replicated formulations were (1) −35±4 mV for Cit-Ghost-CPSNPs and (3) −36±4 mV for Cit-FdUMP-CPSNPs. After surface modification, the zeta potential magnitude is depressed by the presence of the mPEG coating in (2) mPEG-Ghost-CPSNPs and (4) mPEG-FdUMP-CPSNPs, which were −1±1 mV and −5±4 mV, respectively.

FIG. 5 is a graph showing the in vitro proliferation of human pancreatic cancer cell lines BxPC-3 and PANC-1 after no treatment (NT), treatments with PBS vehicle, and 200 μM of free 5FU and FdUMP. While both 5FU and FdUMP were equally effective in decreasing BxPC-3 proliferation, the equivalent dose of FdUMP was twice as effective as 5FU in reducing PANC-1 proliferation, *p=0.01.

FIG. 6 are graphs showing a greater pancreatic cancer cell knock-down effect by mPEG-FdUMP-CPSNPs than the free, unencapsulated drug. The in vitro proliferation of human pancreatic cancer cell lines BxPC-3 and PANC-1, human pancreatic ductal epithelial cell line H6c7, and human pancreatic stellate cell line RLT-PSC was measured at 72 hours after (A) no treatment, (B) PBS vehicle, treatment with (C) 200 μM free FdUMP, (D) mPEG-Ghost-CPSNPs, and mPEG-FdUMP-CPSNPs in decreasing doses, (E1) 21 μM, (E2) 840 nM, (E3) 420 nM, (E4) 280 nM, and (E5) 210 nM. MPEG-FdUMP-CPSNPs blocked the growth of pancreatic cancer cells and stellate cells while having a lesser effect on normal human pancreatic ductal cell proliferation. Proliferation was normalized to the vehicle control for each cell line.

FIG. 7 are a series of graphs compared side by side showing in vitro growth of human PDAC cell lines BxPC-3 and PANC-1 is effectively blocked by mPEG-CPSNPs containing gemcitabine monophosphate (dFdCMP), with EC₅₀ values of 130 and 550 nM, respectively. BxPC-3 cells were more resistant to mPEG-FdUMP-CPSNPS than PANC-1 cells, which had an EC₅₀ of 1.3 μM. Empty CPSNPs (light hatched bars), free drug (dark bars) or drug-containing CPSNPs (dark hatched bars) are expressed as relative proliferation (percent of vehicle controls, white bars). Values are the mean of 3-4 independent experiments with *=p<0.001 and **=p<0.01.

FIG. 8 are two gels of the FdUMP target enzyme thymidylate synthase (TS) from PANC-1 (upper panel) or BxPC-3 (lower panel) cells treated with 250 μM free FdUMP (Lane 3) or 2 μM mPEG-FdUMP-CPSNPs (Lane 5). Both cell lines showed significant (>80%) conversion of TS to an inactive ternary complex (TS:FdUMP) with free drug and with mPEG-FdUMP-CPSNP treatment. Controls that received no treatment (Lane 1), PBS vehicle (Lane 2) or mPEG-CPSNPs containing no FdUMP (Lane 4) exhibited only active TS with no evidence of TS:FdUMP ternary complex formation.

FIG. 9 are graphs showing the arrest of PANC-1 cell progression through the cell cycle by mPEG-FdUMP-CPSNPs. Cell cycle phase was assessed by flow cytometric analysis of propidium iodide-labeled PANC-1 cells treated for 72 hr with mPEG-FdUMP-CPSNPs. Treatment groups included cells treated with the PBS vehicle, no treatment, 250 uM free FdUMP, mPEG-Ghost-CPSNPs, and 200 nM mPEG-FdUMP-CPSNPs. Arrowheads indicate G0/G1 and G2/M peaks, in red, while S phase cells are indicated with hatch marks.

FIG. 10 is a graph showing the in vitro evaluation of metastatic colon cancer cell lines, LoVo, HCT116, and SW620, and pancreatic cancer, PANC-1, after a 72 hr treatment with the PBS vehicle, free Gem, mPEG-Ghost-CPSNP control, and mPEG-GemMP-CPSNPs at various doses. A similar response in reduced cell proliferation is observed with 125 nM of mPEG-Ghost-CPSNPs compared with 500 nM of the free non-phosphorylated Gem.

FIG. 11 is a graph showing CCKBR-targeted CPSNPs deliver active FdUMP to PDAC tumors in vivo. Levels of active thymidylate synthase (unbound TS) was determined by immunoblotting, and reflects that amount of the TS inhibitor FdUMP taken up by PANC-1 tumors in mice treated with various CPSNP formulations (n=5 mice/treatment group). Tumors from mice treated with empty (non-drug containing) CPSNPs (#1, black bar) or untargeted mPEG-FdUMP-CPSNPs (#2, grey bar) had equivalent amounts of unbound, active TS, suggesting that untargeted particles were not efficiently taken up by tumor cells in vivo. Although the mean TS levels in tumors from gastrin-16 peptide targeted-mPEG-FdUMP-CPSNPs treated mice was decreased (#4, light hatched), only tumors in mice treated with CCKBR aptamer targeted mPEG-FdUMP-CPSNPs (#3, dark hatched) had significantly reduced TS levels (*p<0.05) compared to empty CPSNP or untargeted mPEG-FdUMP-CPSNP controls. Bars represent ±SEM of 2 independent experiments.

### DETAILED DESCRIPTION

The present invention now will be described more fully hereinafter with reference to the accompanying examples, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout. References referred to herein are incorporated by reference in their entirety.

Many modifications and other embodiments of the invention set forth herein will come to mind to one skilled in the art to which this invention pertains, having the benefit of the teachings presented in the descriptions and the drawings herein. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The articles “a” and “an” are used herein to refer to one or more than one (i.e., to at least one) of the grammatical object of the article. By way of example, “an element” means one or more than one element.

Based on numerous fundamental studies on the binding of calcium to adenosine triphosphate to form ionic Ca-ATP complexes 44,45,46,47, an embodiment here demonstrates that high encapsulation efficiencies can be achieved via adsorption of the negatively charged phosphate groups to calcium sites as CPSNPs are formed in the reverse micelles by an agglomerative-growth process.

The methods described herein can be utilized with any convenient drug or imaging agent. When referring to a drug is meant a substance or compound that can be used in the diagnosis, treatment or prevention of a disease or as a component of a medication. Imaging agents are compounds designed to allow improved imaging of specific organs, tissues, tumors, diseases or physiological functions within a mammalian body.

Additionally, the nanoparticles used in the invention may encapsulate other agents, including those useful in the treatment of tumors. Preferred agents include drugs, apoptosis inducers such as bioactive lipids, including ceramide or dihydroceramide, DNA, plasmids, shRNA, siRNA, antineoplastic chemotherapeutics, other agents that useful in inhibiting or treating tumors.

Optionally, the nanoparticles used in the invention may be conjugated to various ligands or antibodies to facilitate targeting to the target tissue. These ligands include those that are receptor-specific as well as immunoglobulins and fragments thereof. Preferred ligands include antibodies in general and monoclonal antibodies, as well as immunologically reactive fragments of both including antiCD71 and transferrrin for breast cancer, gastrin and penta-gastrin for pancreatic and colon cancer, antiCD 151 for melanoma and similar targets for other cancerous tumors.

The nanoparticles may be PEGylated for surface polyethylene glycol (PEG) functionalizaiton to facilitate their accumulation in tumors. (Altinoglu et al. (2008) Near-infrared emitting fluorophore-doped calcium phosphate nanoparticles for in vivo imaging of human breast cancer. ACS Nano. 2: 2075-2084.). See Example 16. In a preferred embodiment, ICG has been encapsulated into PEGylated CPNP's. See the Examples disclosed herein.

In an embodiment the active phosphorylated forms of the drug agents, FdUMP and GemMP, are being delivered by CPSNPs, which are biologically more potent that their non-phosphorylated prodrug counterparts, 5FU and GEM. Encapsulated drug agents have higher efficacy than the delivery of the free form in vitro on the cancer cells, in this example, on human pancreatic and colon cancers. The delivered drug dose by CPSNPs is not toxic to healthy normal cells.

Phosphorylated drugs were believed to be almost immediately cleared by the immune system in vivo and thus expectations are there would be little efficacy for treatment of diseases including human cancer. However, here therapeutic levels of the drugs and imaging agents are encapsulated and delivered to human concerns and cancer cells and overcomes both poor encapsulation efficiency and poor efficacy of phosphorylated drugs and imaging agents.

In certain embodiments, the encapsulated drugs have increased efficacy of at least 10%, 20%, 30%, 40%, 50%, 60%, 70%, 80%, 90%, 100% or more or amounts in-between compared to nonencapsulated forms of the drug. The efficacy in one embodiments can be measured by reduction in cancer cell proliferation, in vitro or in vivo. The encapsulated drug or imaging agent can in an embodiment have the same efficacy as a nonencapsulated form of the drug, but at a dose that is up to 1000× less than the nonencapsulated form of the drug or imaging agent. The ability of the encapsulated drug can in a stiff further embodiment arrest cancer cells at a critical phase of development, and in another embodiment can arrest cancer cells prior to G1 phase.

The calcium phosphosilicate and calcium phosphate nanoparticle systems are efficacious at delivering drugs or imaging agents encapsulated within the particles. In one instance encapsulation efficiency is defined as the amount of drug or imaging agent introduced at the beginning of the synthesis divided into the amount of drug or imaging agent encapsulated in the nanoparticles. Previously, the best encapsulation efficiency achieved was 2%. The encapsulation efficiency achieved with the phosphorylated drugs and imaging agents ins in the range of at least 10%, and can be 30%, 40%, 50%, 60%, 70%, 80%, 90%, up to 100%.

Unlike surface decorated nanoparticles that are also tailored for drug delivery, the drug molecules are located within the CPSNP matrix, which protects the agents from metabolic breakdown by liver enzymes. This ensures that the drug is able to reach the tumor and reduce off-target toxicities. The nanoparticle carrier material, calcium phosphate, is inherently non-toxic to cells and degraded products are bioresorbable.

Accordingly, the compositions and methods of the present invention can be used to treat a variety of cancer cells of mammalian tumors. As used herein, the term “treating” refers to: (i) preventing a disease, disorder or condition from occurring in a mammal, animal or human that may be predisposed to the disease, disorder and/or condition but has not yet been diagnosed as having it; (ii) inhibiting the disease, disorder or condition, i.e., arresting its development; and/or (iii) relieving the disease, disorder or condition, i.e., causing regression of the disease, disorder and/or condition. For example, with respect to cancer, treatment may be measured quantitatively or qualitatively to determine the presence/absence of the disease, or its progression or regression using, for example, reduction in tumor size, a reduction in the rate of metastasis, and/or a slowing of tumor growth, and/or no worsening in disease over a specified period of time or other symptoms associated with the disease or clinical indications associated with the pathology of the cancer.

As used herein, the term “nano-encapsulated” refers to enclosing or embedding the photosensitizer within a nano matrix, e.g. within a nanoparticle. The photosensitizer may be encapsulated within any suitable matrix of a nanoparticle. As used herein, the term “nanoparticle” includes a nanosphere and/or a nanocolloid. In a preferred embodiment, the nanoparticle comprises a calcium phosphate matrix to produce a calcium phosphate nanoparticle (CPNP). CPNP includes nano-sized calcium phosphate-based composite particles. It is preferred that the nanoparticles are composed of non-toxic, resorbable compounds that are colloidally stable in physiological fluids or solutions, those solutions having a pH around 7.4. e.g. phosphate buffered 0.15M saline. Physiological fluid includes but is not limited to, blood, cerebrospinal fluid, interstitial fluid, semen, sweat, saliva, urine and the like. The term colloidally stable refers to nanoparticles that are non-agglomerated, able to form a uniform and stable suspension of solids in solution or combinations thereof. As used herein, the term “nano” in reference to nanoparticles refers to nanoparticles that are less than 200 nm in diameter and typically less than 60 nm. Typically, the encapsulated nanoparticles preferably have a mean diameter of less than about 200 nm, more preferably between about 10 nm and about 75 nm, with the preferred particle diameter range between about 15 to about 25 nm. The size of nanoparticles can be measured by a number of means known in the art for sizing small particles, including the use of a Malvern Zetasizer, Nanosight NTA system, Nicomp™ particle sizer or a Coulter™ Nano-Sizer (Coulter Electronics, Harpenden, Hertfordshire, UK). Without wishing to be bound by this theory, it is believed that particles greater than 200 nm interact with organics in the bloodstream to agglomerate and do not survive the immune response.

Various methods of preparing nanoparticles for encapsulating agents may be used. By way of example without limitation, see Adair et al, U.S. Pat. No. 8,771,741, incorporated herein by reference in its entirety. See also for further examples, Altinoglu, E., et al. “Near-Infrared Emitting Fluorophore-Doped Calcium Phosphate Nanoparticles for in Vivo Imaging of Human Breast Cancer.” ACS Nano 2.10 (2008): 2075-84.). CPNPs or CPNSPs are both referred to here, and where referring to CPNPs also applies to CPNSPs. The nanoparticles may be prepared using any suitable technique, for example, from a controlled addition to a phosphate solution to a calcium solution to the use of a double microemulsions as templates for particle size. (Bisht, S.; Bhakta, G.; Mitra, S.; Maitra, A. International Journal of Pharmaceutics 2004, 288, 157-168; Welzel, T.; Radtke, I.; Meyer-Zaika, W.; Heumann, R.; Epple, M. Journal of Materials Chemistry 2004, 14, 2213-2217; Sadasivan, S.; Khushalani, D.; Mann, S. Chemistry of Materials 2005, 17, 2765-2770; Sarda, S.; Heughebaert, M.; Lebugle, A. Chemistry of Materials 1999, 11, (2722-2727). Preferably, methods utilized to prepare nanoparticles of the invention produce colloidally stable nanoparticles with diameters less than 100 nm that are well-dispersed and avoid agglomeration in physiological fluids. The CPNPs or CPNSPs are colloidally stable in a wide range of solutions including phosphate saline (10 mM phosphate buffered to pH 7.4, 0.14M NaCl, 0.01M KCl) and various ethanolic solutions used in the processing. More importantly, the polyethylene glycol surface conjugated CPNPs demonstrated colloidal stability and the ability to remain in the circulatory of a nude mouse model for at least 96 hours, verifying the colloidal stability and non-agglomerating characteristics in this animal model.

Exemplary detailed methods for preparing CPNP's are described elsewhere herein. Briefly, the general synthesis scheme of organically-doped, functionalized calcium phosphate nanoparticles (CPNPs) was adapted from recently published silica syntheses. Wang, J.; White, W. B.; Adair, J. H. Journal of American Ceramic Society 2006, 89, (7), 2359-2363; Wang, J.; White, W. B.; Adair, J. H. Journal of Physical Chemistry B 2006, 110, 4679-4685; Li, T.; Moon, J.; Morrone, A. A.; Mecholsky, J. J.; Talham, D. R.; Adair, J. H. Langmuir 1999, 15, 4328-4334. It is preferred that the nanoparticles are prepared using van der Waals chromatography.

Drug or imaging agent—encapsulated CPNP's may be formulated in any suitable manner. In some examples, the CPNP's comprising the drug or imaging agent are conveniently formulated as sterile, freeze-dried powders containing trehalose or another lyoprotectant. The drug or imaging agent encapsulated nanoparticles are conveniently formulated as sterile, freeze-dried powders containing trehalose or another lyoprotectant. A typical powder can contain a lyoprotectant/nanoparticle ratio in the range of about 0.1 to about 5, preferably in the range of about 0.6 to 3.0, and more preferably in the range of about 0.8 to 2.0 on a weight/weight basis. A sterile freeze-dried power containing nanoparticles and optional lyoprotectant may be reconstituted in an aqueous medium for administration to a human or other animal. The aqueous medium is preferably a pharmaceutically acceptable sterile medium, for example 5% dextrose or normal saline. Alternatively, the medium may be water for injection where the amount of lyoprotectant or other additive is sufficient to render the reconstituted material suitable for pharmaceutical or therapeutic use.

The nanoparticles of the invention may be formulated into a variety of additional compositions. These compositions may also comprise further components, such as conventional delivery vehicles and excipients including isotonising agents, pH regulators, solvents, solubilizers, dyes, gelling agents and thickeners and buffers and combinations thereof. Suitable excipients for use with photosensitizers include water, saline, dextrose, glycerol and the like.

Appropriate formulations and dosages for the administration of the drugs or imaging agents are known in the art. The particular concentration or amount of a given drug or imaging agent is adjusted according to its intended use. Referring to Adair, U.S. Pat. No. 8,771,741, it was noted that CPNPs are colloidally stable in a wide range of solutions including phosphate saline (10 mM phosphate buffered to pH 7.4, 0.14M NaCl, 0.01M KCl) and various ethanolic solutions used in the processing. The polyethylene glycol surface conjugated CPNPs demonstrated colloidal stability and the ability to remain in the circulatory of a nude mouse model for at least 96 hours, verifying the colloidal stability and non-agglomerating characteristics in this animal model. Accordingly, contrary to conventional teachings of colloidal chemistry, calcium phosphate nanoparticles encapsulating drugs or imaging agents having citrate, amine or PEG surface functionalized particles are stable in PBS and do not form agglomerates. Suitable isotonising agents are preferably nonionic isotonising agents such as glycerol, sorbitol, mannitol, aminoethanol or propylene glycol as well as ionic isotonising agents such as sodium chloride. The solutions of this invention will contain the isotonising agent, if present, in an amount sufficient to bring about the formation of an approximately isotonic solution. The expression “an approximately isotonic solution” will be taken to mean in this context a solution that has an osmolarity of about 300 milliosmol (mOsm), conveniently 300.+−0.10% mOsm. It should be borne in mind that all components of the solution contribute to the osmolarity. The nonionic isotonising agent, if present, is added in customary amounts, i.e., preferably in amounts of about 1 to about 3.5 percent by weight, preferably in amounts of about 1.5 to 3 percent by weight. Summaries of pharmaceutical compositions suitable for use with photosensitizers are known in the art and are found, for instance, in Remington's Pharmaceutical Sciences.

As mentioned above, compositions and methods of the present invention may be used in imaging of target tissue or tumors, to treat any number of cancers or tumors or both. The nanoparticles here described are particularly suited for the imaging and/or treatment of deep tissue tumors, such breast cancer, ovarian cancer, brain cancer, lung cancer, hepatic cancers, and the like. Types of mammalian tumors that can be treated using the compositions and methods of the present invention include, but are not limited to all solid tumors, cutaneous tumors, melanoma, malignant melanoma, renal cell carcinoma, colorectal carcinoma, colon cancer, hepatic metastases of advanced colorectal carcinoma, lymphomas (including glandular lymphoma), malignant lymphoma, Kaposi's sarcoma, prostate cancer, kidney cancer, ovarian cancer, lung cancer, head and neck cancer, pancreatic cancer, mesenteric cancer, gastric cancer, rectal cancer, stomach cancer, bladder cancer, leukemia (including hairy cell leukemia and chronic myelogenous leukemia), breast cancer, solid breast tumor growth, non-melanoma skin cancer (including squamous cell carcinoma and basal cell carcinoma), hemangioma multiple myeloma, and glioma. The cancer in an embodiment is brain, breast, lung, pancreatic, hepatic, colon, melanoma, ovarian cancer, or metastases thereof. In addition, embodiments for the invention can be adapted for non-solid tumors.

In one aspect, the methods include administering systemically or locally the drug or imaging agent-encapsulated nanoparticles of the invention. Any suitable route of administration may be used, including, for example, topical, intravenous, oral, subcutaneous, local (e.g. in the eye) or by use of an implant. Advantageously, the small size, colloidal stability, non-agglomeration properties, and enhanced half-life of the nanoparticles renders the nano-encapsulated drug or imaging agent especially suitable for intravenous administration. Additional routes of administration are subcutaneous, intramuscular, or intraperitoneal injections in conventional or convenient forms. For topical administration, the nanoparticles may be in standard topical formulations and compositions including lotions, suspensions or pastes.

The dose of nanoparticles may be optimized by the skilled person depending on factors such as, but not limited to, the drug or imaging agent chosen, the nature of the therapeutic protocol, the individual subject, and the judgment of the skilled practitioner. Preferred amounts of nanoparticles are those which are clinically or therapeutically effective in the treatment method being used. Such amounts are referred herein as “effective amounts”.

Depending on the needs of the subject and the constraints of the treatment method being used, smaller or larger doses of nanoparticles may be needed. The doses may be a single administration or include multiple dosings over time. For compositions which are highly specific to the target skin tissues and cells, such as those with the nanoparticles conjugated to a highly specific monoclonal antibody preparation or specific receptor ligand, dosages in the range of 0.005-10 mg/kg of body weight may be used. For compositions, which are less specific to the target, larger dosages, up to 1-10 mg/kg, may be desirable. One preferred range for use in mice is from 0.05 mg/kg to 10 mg/kg. The useful range in humans for the photosensitizer-encapsulated nanoparticles will generally be lower than mice, such as from 0.005 mg/kg to 2 mg/kg. The foregoing ranges are merely suggestive in that the number of variables with regard to an individual treatment regime is large and considerable deviation from these values may be expected. The skilled artisan is free to vary the foregoing concentrations so that the uptake and stimulation/restoration parameters are consistent with the therapeutic objectives disclosed above.

In addition to human subjects, the present invention may be applied to non-human animals, such as mammals, particularly those important to agricultural applications (such as, but not limited to, cattle, sheep, horses, and other “farm animals”), industrial applications (such as, but not limited to, animals used to generate bioactive molecules as part of the biotechnology and pharmaceutical industries), and for human companionship (such as, but not limited to, dogs and cats).

The nanoparticles are administered to the subject and the tumor or tissue or cell in the subject is exposed for a sufficient amount of time to achieve the desired results, as in being able to bioimage or detect the target tissue or tumor or to reduce tumor growth or size or reduce cell proliferation.

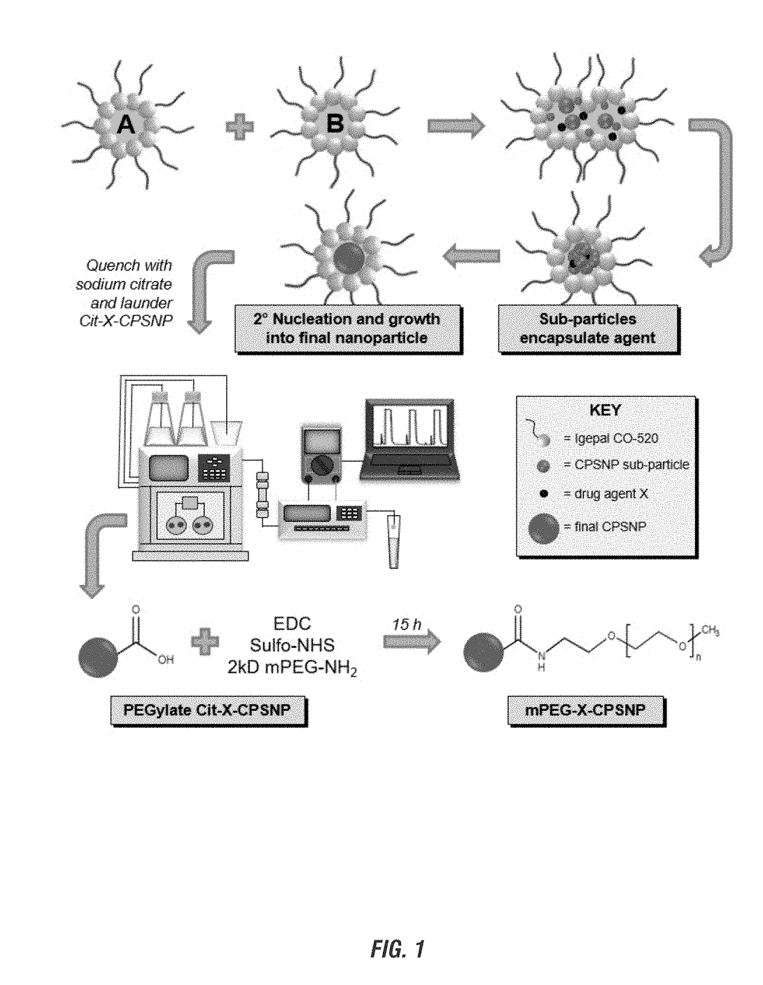

FIG. 1 shows a schematic of the calcium phosphosilicate nanoparticle double reverse-micelle synthesis and encapsulation procedure. Micelle water pool size is maintained by the water-to-surfactant mole ratio, ω=[H2O]/[Igepal CO-520]=4. Microemulsions A and B consist the aqueous calcium chloride, and hydrogen phosphate/metasilicate/drug precursors, respectively. Immediately following micellar exchange from combining A and B, sub-particles form as drug agents (black dots) adsorb to, or become encapsulated by, the calcium phosphate material. Secondary nucleation and growth complete the nanoparticle and the reaction is quenched with sodium citrate. This Cit-X-CPSNP suspension is laundered with a van der Waals HPLC system equipped with a UV/Vis spectrophotometer to monitor the launder cycles. The laundered nanoparticles are PEGylated to obtain mPEG-X-CPSNPs. X=5FU, 5FU:ATP, FdUMP, FUdR, Gem, GemMP, or Ghost (negative CPSNP control). Micelles and CPSNPs are not drawn to scale.

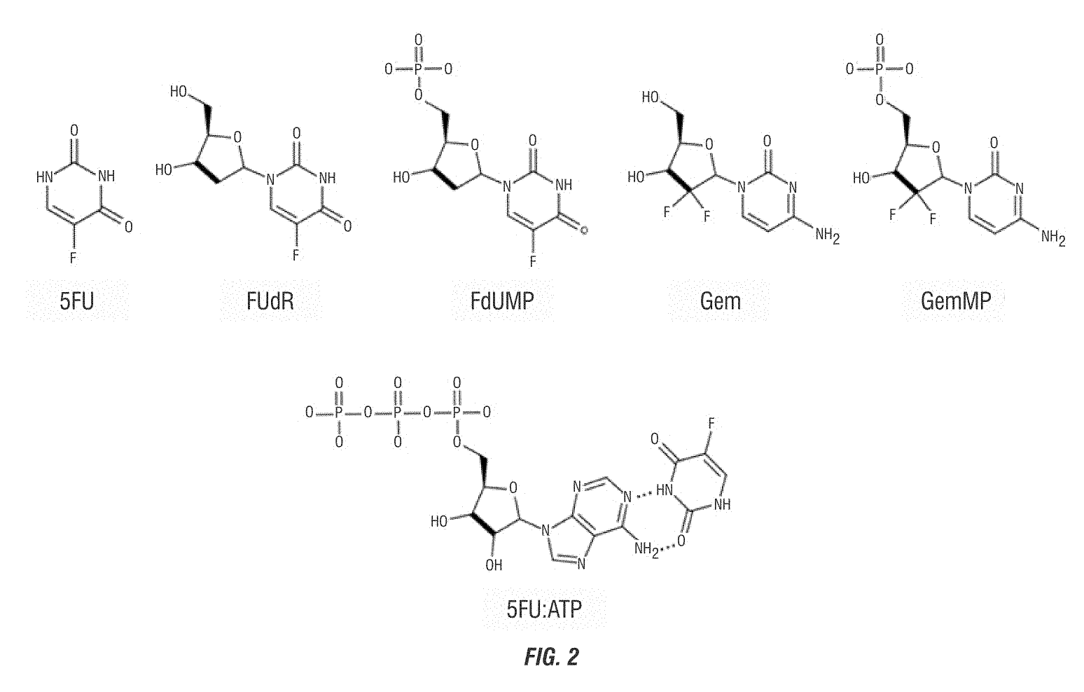

FIG. 2 shows structures of several encapsulated drug agents, 5-fluorouracil (5FU), 5-fluorouracil, adenosine 5′-triphosphate (5FU:ATP), 5-fluoro-2′-deoxyuridine (FUdR), 5-fluoro-2′-deoxyuridine monophosphate (FdUMP), gemcitabine (Gem), and gemcitabine monophosphate (GemMP).

The delivery of chemotherapeutic drug agents by nanoparticles offers an alternative method from chemotherapy to treat highly drug resistant and fibrotic cancers like pancreatic ductal carcinoma (PDAC). This study investigated whether 5-fluoro-2′-deoxyuridine 5′-monophosphate (FdUMP), an active metabolite of 5-fluorouracil (5FU), can be encapsulated at therapeutic doses and retain biological activity in calcium phosphosilicate nanoparticles (CPSNPs). CPSNPs were synthesized to encapsulate FdUMP, 5FU or 5-fluoro-2′-deoxyuridine (FUdR), and surface-modified with methoxy-terminated polyethylene glycol (mPEG). LC-MS/MS analysis showed that only the phosphorylated agent, FdUMP, was encapsulated successfully and not the non-phosphorylated 5FU and FdUR. The encapsulated concentration of FdUMP in CPSNPs was 6.0×10⁻⁵-8.3×10⁻⁴M at 7-97 mol % encapsulation efficiency in replicated formulations. Free FdUMP was twice as effective as free 5FU in reducing PANC-1 proliferation in vitro, and the encapsulation of FdUMP within CPSNPs further improved efficacy. For BxPC-3 and RLT-PSC cells, treatment with 210 nM mPEG-FdUMP-CPSNPs was as cytotoxic as 200 μM free FdUMP, a dose that was at least 1000 times less, while leaving non-transformed H6c7 cells less affected by treatment. Cell cycle analysis demonstrated that treatment with mPEG-FdUMP-CPSNPs arrested cancer cells prior to entrance into G1 phase. Western blots revealed successful inhibition of thymidylate synthase, which indicated that the FdUMP in CPSNPs remained biologically active to block cell proliferation. Thus, the findings reported herein represent a novel methodology to encapsulate and deliver an efficacious dose of active agents to pancreatic cancer without inducing non-specific toxicity.

Lipid-coated calcium-phosphate particles can successfully encapsulate and deliver gemcitabine monophosphate, gemcitabine triphosphate,²³ and siRNAs^{24, 25, 26} to a variety of tumor types, the proposed CPSNP platform in this study does not require an additional lipid coating to achieve colloidal stability due to the exposed surface carboxylate functional groups that can also be readily modified to target cancer cell receptors. The inventors hypothesized that chemotherapeutic drugs such as 5-FU or gemcitabine (dFdC) would be less effectively encapsulated into CPSNPs than their bioactive nucleotide analogs FdUMP and dFdCMP.

This present work also explicitly focuses and compares the encapsulation efficiency of both phosphorylated and non-phosphorylated agents, which involves quantitative analysis of drug concentration in the particles by a LC-MS/MS protocol specially designed for this purpose. The development of a novel approach to delivering the cytotoxic chemotherapeutic FdUMP by encapsulation of this active 5FU metabolite in CPSNPs and the efficacy of particles with FdUMP against human pancreatic cancer cells in vitro are also described.

The following is presented by way of exemplification and is not intended to limit the scope of the invention.

### EXAMPLES

### Example 1

### Encapsulation Efficiencies of CPSNPs

The encapsulation efficiencies (EE) in Table 1 was experimentally determined by LC-MS/MS for 5FU, 5FU:ATP, FUdR, FdUMP, Gem, and GemMP, as drawn in FIG. 2 1. The EE is defined by the following equation,

$$mol \% EE = \frac{{m}_{f}}{{m}_{i}} \times 100$$

where mᵢ was the total drug content in moles and m_{f} was the moles encapsulated as assessed by LC-MS/MS. Particles were diluted in 10% methanol with 0.1% formic acid and 5-CU was spiked in as an internal standard. Chromatography was done on a 2.1 mm×10 cm HSS T3 or C18 CSH column (Waters) on a Waters I-class FTN chromatography system with the column temperature at 40° C. Mobile phase A was water with 5 mM ammonium acetate and B was methanol. The flow rate was 0.5 mL/min and the chromatography consisted of holding at 7.5% B for 1 min, increasing to 95% B over 0.5 min, holding at 95% B for 0.5 min before equilibration to starting conditions. Eluate was analyzed by an inline Waters TQ-S mass spectrometer. The capillary was set at 1.0 kV, source temperature at 150° C., desolvation temperature at 600° C., cone gas at 150 L/hr, and desolvation gas flow at 1200 L/hr. Multiple reaction monitoring (MRM) was used to detect 5-CU, FdUMP, Gem, and GemMP. The MRM transition used for 5-CU was 145 >42 with the collision energy set at 12. The MRM transition used for drug agents was 325 >195 with the collision energy set at 12. Drug concentrations were determined using TargetLynx version 4.1 (Waters) using an external calibration curve with 1/x weighting.

Both centrifugation and van der Waals HPLC laundering methods were used in early attempts at encapsulating 5FU. The micellar exchange time was also increased from 2 min (5FU-1, 2, 3) to 30 min (5FU-4, 5, 6) in effort to encapsulate more drug molecules. Although the goal was to encapsulate an effective dose of 5FU in CPSNPs, only 1.7 to 7.6×10⁻⁷ M 5FU, or 0.07 to 0.2 mol % EE, uptake was achieved. See Table 1.

TABLE 1
A
Encapsulated drug concentration and encapsulation efficiency (EE) of
non-phosphorylated agents, 5FU, 5FU:ATP, FUdR, Gem, and
phosphorylated agents, FdUMP and GemMP, in Cit-X-CPSNPs.
Simplify table, give average and confidence interval for n = x. With
average and confidence interval, this table goes into patent application.

| | Encapsulated | |
|---|---|---|
| Formulation no. | drug concentration (M) | EE (mol %) |
| Cit-5FU-CPSNP | | |
| 5FU-1 | 1.700 ± 0.002 × 10⁻⁷ | 0.08 ± 0.01 |
| 5FU-2 | 2.100 ± 0.003 × 10⁻⁷ | 0.11 ± 0.01 |
| 5FU-3 | 3.600 ± 0.005 × 10⁻⁷ | 0.10 ± 0.01 |
| 5FU-4 | 7.60 ± 0.01 × 10⁻⁷ | 0.21 ± 0.03 |
| 5FU-5 | 5.80 ± 0.01 × 10⁻⁷ | 0.12 ± 0.02 |
| 5FU-6 | 3.900 ± 0.005 × 10⁻⁷ | 0.07 ± 0.01 |
| Cit-5FU:ATP-CPSNP | | |
| 5FU:ATP-1 | 1.400 ± 0.002 × 10⁻⁶ | 0.93 ± 0.12 |
| 5FU:ATP-2 | 1.900 ± 0.002 × 10⁻⁷ | 0.01 ± 0.01 |
| 5FU:ATP-3 | 2.200 ± 0.003 × 10⁻⁷ | 0.15 ± 0.02 |
| 5FU:ATP-4 | 5.50 ± 0.01 × 10⁻⁶ | 0.37 ± 0.05 |
| Cit-FUdR-CPSNP | | |
| FUdR-1 | 5.0 ± 3.0 × 10⁻⁸ | <0.01 |
| FUdR-2 | 3.0 ± 1.5 × 10⁻⁸ | <0.01 |
| FUdR-3 | 2.8 ± 2.2 × 10⁻⁸ | <0.01 |
| FUdR-4 | 2.1 ± 2.0 × 10⁻⁸ | <0.01 |
| FUdR-5 | 1.1 ± 0.7 × 10⁻⁸ | <0.01 |
| Cit-Gem-CPSNP | | |
| Gem-1 | 8.7 ± 0.1 × 10⁻¹⁰ | <0.01 |
| Gem-2 | 2.10 ± 0.04 × 10⁻⁹ | <0.01 |
| Gem-3 | 1.80 ± 0.02 × 10⁻⁹ | <0.01 |
| Gem-4 | 2.90 ± 0.08 × 10⁻⁹ | <0.01 |
| Gem-5 | 2.00 ± 0.06 × 10⁻⁹ | <0.01 |
| Cit-FdUMP-CPSNP | | |
| FdUMP-1 | 5.5 ± 0.2 × 10⁻⁵ | 7 ± 0.2 |
| FdUMP-2 | 1.30 ± 0.03 × 10⁻⁴ | 21 ± 1 |
| FdUMP-3 | 3.50 ± 0.07 × 10⁻⁴ | 57 ± 1 |
| FdUMP-4 | 8.30 ± 0.02 × 10⁻⁴ | 97 ± 0.3 |
| Cit-GemMP-CPSNP | | |
| GemMP-1 | 2.40 ± 0.03 × 10⁻⁵ | 24 ± 0.3 |
| GemMP-2 | 3.00 ± 0.03 × 10⁻⁵ | 28 ± 0.3 |
| GemMP-3 | 1.90 ± 0.05 × 10⁻⁵ | 15 ± 0.4 |
| GemMP-4 | 2.00 ± 0.01 × 10⁻⁵ | 15 ± 0.1 |
| GemMP-5 | 2.20 ± 0.04 × 10⁻⁵ | 30 ± 0.5 |

B
Encapsulated concentrations and encapsulation efficiency (EE) for
5-FU, 5-FU:ATP, FUdR, FdUMP, dFdC (gemcitabine) or dFdCMP
(gemcitabine monophosphate) in citrate-functionalized CPSNPs.

| | Drug | |
|---|---|---|
| Formulation | concentration (M)* | EE (mol %)* |
| Cit-5-FU-CPSNP | 4.1 (±1.8) × 10⁻⁷ | 0.11 (±0.04) |
| Cit-5-FU:ATP-CPSNP | 1.8 (±2.5) × 10⁻⁶ | 0.36 (±0.39) |
| Cit-FUdR-CPSNP | 2.8 (±1.2) × 10⁻⁸ | <0.01 |
| Cit-FdUMP-CPSNP | 3.0 (±1.4) × 10⁻⁴ | 41 (±16) |
| Cit-dFdC-CPSNP | 1.9 (±0.6) × 10⁻⁹ | <0.01 |
| Cit-dFdCMP-CPSNP | 2.3 (±0.4) × 10⁻⁵ | 22 (±6) |

*Concentration and encapsulation efficiency (EE) are expressed as the weighted mean ± 95% confidence interval of n = 4-6 experimental replicates.

CPSNP encapsulation of 5-FU was only 4.1 (±1.8)×10⁻⁷M, or 0.11 (±0.04) mol % EE (Table 1B), and extending micellar exchange times failed to improve 5-FU encapsulation. FUdR, the deoxynucleoside analog of 5-FU, was incorporated in CPSNPs even less effectively than 5-FU, with an EE of <0.01 mol % and 2.8 (±1.2)×10⁻⁸M encapsulated drug. When a mixture of 5-FU and ATP was used for encapsulation, the EE increased only slightly to 0.36 (±0.39) mol %.

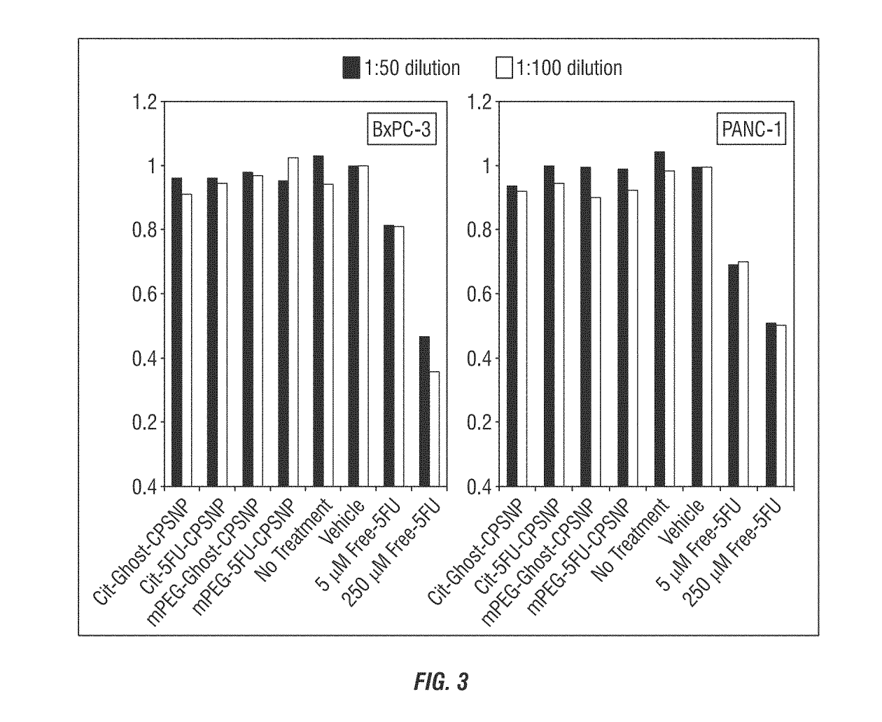

This is an amount of uptake that was not sufficient for a therapeutic effect when tested in vitro on both BxPC-3 and PANC-1 cell lines, as shown in FIG. 3 example with both 250 μM and 5 μM free 5FU as reference points (the 1:50 dilution is the left bar, the 1:100 dilution is the right bar). Using a pre-mixed precursor solution of 5FU and ATP, the EE increased to nearly 1 mol %. A triple microemulsion method was implemented to create a core-shell CPSNP, which consists a calcium phosphosilicate (CPS) core with 5FU:ATP and an additional CPS shell to bring the final water-to-surfactant ratio to 4. The incorporation of ATP utilizes the hydrogen bonds between the fluorouracil group and adenine ring, so that the phosphate tail can become incorporated with 5FU for adsorption-mediated encapsulation. However, these weak hydrogen bonds were compromised once introduced to the high ionic strength environment of micelles during synthesis. Thus, this method was not easily reproducible and 0.93 mol % was the highest EE that was achieved out of 5FU:ATP-1,2,3, and 4.

FudR is 5FU with an extra ribose ring and can either be rapidly cleaved to 5FU or phosphorylated into FdUMP in vivo by enzymes. FudR was incorporated in CPSNPs at <0.01 mol % EE, corresponding to only 1.1 to 5.0×10⁻⁷ M of encapsulated drug in five repeated formulations, FudR-1, 2, 3, 4, and 5. Both the encapsulation of 5FU and FudR were relatively unsuccessful compared to FdUMP.

FdUMP was reproducibly encapsulated into CPSNPs from 6.0×10⁻⁵ to 8.3×10 M, which revealed the significance of the phosphate group for successful encapsulation (FdUMP-1, 2, 3, 4). This concentration was ˜100× higher than encapsulated 5FU. The EE of Cit-FdUMP-CPSNPs was 7 to 97 mol % with batch-to-batch variations influenced by the laundering procedure. In contrast to the low encapsulation efficiencies of 5-FU and FudR, FdUMP was reproducibly encapsulated into CPSNPs at 3.0 (±1.4)×10⁻⁴ M and an EE of 41 (±16) mol %−several orders of magnitude higher than 5-FU or FudR encapsulation (Table 1). The recovery of FdUMP after PEGylating the Cit-FdUMP-CPSNPs was 30 (±9) mol %, in part due to the binding of particles to the filter membrane. The increased EE for FdUMP versus FudR suggests that encapsulation is enhanced by the formation of a metal-ligand bond between calcium and phosphate (see Supporting Information Figures S2 and S3 for further discussion). Similarly, free gemcitabine (dFdC) was poorly encapsulated into CPSNPs, 1.9 (±0.6)×10⁻⁹ M with an EE of <0.01 mol %, while gemcitabine monophosphate (dFdCMP) was encapsulated with significantly higher efficiency, 2.3 (±0.4)×10⁻⁵ M and an EE of 22 (±6) mol %. Thus, while the standard chemotherapeutics 5-FU and gemcitabine were not well encapsulated into CPSNPS, we have demonstrated successful encapsulation of the bioactive phosphodrugs FdUMP and dFdCMP into mPEG-CPSNPs.

Changes in the internal flow rate of the HPLC and the amount of fractions collected per elution cycle contributed to this wide range of EE obtained. Thus, the yield consistency can be further improved in future work by reducing the loss of particles during laundering such as increasing the loading capacity of the column or shortening the loading time.

The concentration of FdUMP after surface modification, summarized in Table 2, was comparable to the corresponding Cit-FdUMP-CPSNPs from Table 1, 2.0 to 6.4×10⁻⁴M.

TABLE 2
FdUMP, Gem, and GemMP concentration and percent recovery in
PEGylated Cit-X-CPSNPs. Combine with Table 1, but give averages and
confidence intervals under subheadings; as-prepared with Citrate and then

| | Recovered | |
|---|---|---|
| Formulation no. | drug concentration (M) | Recovery (mol %) |
| mPEG-FdUMP-CPSNP | | |
| FdUMP-2 | 2.0 ± 0.1 × 10⁻⁴ | 48 ± 1 |
| FdUMP-3 | 3.2 ± 0.2 × 10⁻⁴ | 21 ± 2 |
| FdUMP-4 | 5.4 ± 0.4 × 10⁻⁴ | 22 ± 1 |
| FdUMP-5 | 6.4 ± 0.2 × 10⁻⁴ | i.d. |
| mPEG-GemMP-CPSNP | | |
| GemMP-1 | 1.8 ± 0.4 × 10⁻⁵ | 75 ± 8 |
| GemMP-2 | 2.2 ± 0.2 × 10⁻⁵ | 83 ± 2 |

i.d., incomplete data (value not calculated)

The recovery from the original Cit-FdUMP-CPSNPs was 22 to 48 mol %, which revealed that at least half of the material was lost from ultracentrifugation despite different surface modifications. Results for surface modified GEMPO₄-CPSNPs that were synthesized more recently yielded improved percent recoveries ranging 60 to 83 mol %, which translates to 1.3 to 2.2×10⁻⁵ M of encapsulated drug. The non-phosphorylated prodrug GEM was below the detection limit in mPEG-GEM-CPSNPs because only 8.7×10⁻¹⁰ to 2.9×10⁻⁹ M was originally present in the Cit-GEM-CPSNPs. Like FdUMP, a higher EE was achieved with GEMPO₄ than with GEM. The encapsulated GEMPO₄ in Table 1 was a narrow range of 1.9 to 3.0×10⁻⁵ M, or 15-30 mol % EE.

### Example 2

### CPSNP Preparation

Reagents to perform the synthesis outlined in FIG. 1 include the HPLC stationary phase, which are solid glass microspheres ˜200 μM in diameter (Spheriglass A-Glass 1922, Potters Industries) soaked in purified water for 48 h and thoroughly rinsed with 10⁻³M HCl (Sigma-Aldrich) and 10⁻³M NaOH (J. T. Baker) solutions before use. The stationary phase was wet-packed in a 5 cm long×⅜″ OD, ¼″ ID polycarbonate tube (McMaster-Carr). General chemicals include Igepal CO-520 (Rhodia Chemical Co.), cyclohexane (Alfa Aesar), 5-fluorouracil (5FU, >99%, Sigma-Aldrich), 5-fluoro-2′-deoxyurdine (FUdR, >98%, Tocris), 5-fluoro-2′-deoxyuridine-5′-monophosphate (FdUMP, ˜85-91%, Sigma-Aldrich), adenosine 5′-triphosphate disodium salt hydrate (ATP, 99%, Sigma-Aldrich), gemcitabine hydrochloride (Gem, ≥99%, Sigma-Aldrich), gemcitabine monophosphate formate salt (GemMP, 95%, TRC Canada) calcium chloride dihydrate (CaCl₂, ≥99%, Sigma-Aldrich), sodium hydrogen phosphate (Na₂HPO₄, ≥99%, Sigma-Aldrich), sodium metasilicate (Na₂SiO₃, Sigma-Aldrich), sodium citrate dihydrate (Cit, ≥99%, Sigma-Aldrich), potassium hydroxide pellets (KOH, J. T. Baker) for pH adjustment, neat ethanol (Koptec). N-(3-dimethylaminopropyl)-N′-ethylcarbodiimide hydrochloride (EDC) was purchased from Sigma-Aldrich and N-hydrosulfosuccimide (Sulfo-NHS) was from Thermo Scientific Pierce. The 2 kD methoxy-polyethylene glycol-amine tether (mPEG) was purchased from JenKem Technology. Reagents were all used without further purification. Aqueous solutions were filtered with 0.2 μm cellulose acetate syringe filters (VWR) before use. Deionized-distilled water was obtained from a High-Q 200PT-SYS (RO/IX2) purification system and purged with pre-purified argon. Purified water was tested for endotoxins (<0.100 EU/mL) using a Charles River Limulus Amebocyte Lysate Endochrome kit.

CPSNPs were prepared by a reverse-micelle water-in-oil (Igepal CO-520/cyclohexane/water) microemulsion method and laundered by van der Waals HPLC as previously reported by the Adair group.¹ In a standard formulation, two microemulsions with 650 μl of 10⁻² M CaCl₂.H₂O_{(aq)} in 14.06 ml of 29 vol % Igepal CO-520/cyclohexane and 65 ul of 6.0×10⁻² M Na₂HPO_{4(aq)}/65 ul of 8.2×10⁻³ M Na₂SiO_{3(aq)}/520 μl of purified water in 14.06 ml of 29 vol % Igepal CO-520/cyclohexane were equilibrated for 15 min with constant stirring at ˜200 rpm. For drug-doped CPSNPs, the 520 μl of purified water used for Ghost-CPSNPs was substituted by an equal volume of drug agent to maintain the micelle aqueous pool size, ω=4. The starting concentration of the 5FU precursor was 6.3×10″ M, 0.1 M for 5FU:ATP, 9.2×10⁻³ M for FdUMP, and 2.5×10⁻³ M for Gem and GemMP. Micellar exchange occurred for 2 min and the reaction was quenched with 225 μl of 10⁻³ M citrate for 15 min. The CPSNPs were released from the micelles by the addition of 50 ml of ethanol (pH ˜7.4-9.0). The suspension was loaded in aliquots onto the HPLC column at a flow rate of 2.5-3 ml/min for 10 min and was monitored by UV-Vis absorption (λₘₐₓ=269-276 nm, set to the wavelength of drug agent or Igepal CO-520). The mobile phases were pH adjusted to pH 7.4-9.0 with KOH to mitigate CPSNP dissolution. Excess surfactant and reagents were laundered from the column for 20-30 min with neat ethanol until the absorption baseline was reached and CPSNP fractions were collected in 70/30 ethanol/water (v/v) for 1 min after elution. The load, launder, and collection cycles were repeated until the entire suspension was fully laundered twice.

Filtered citrate-functionalized CPSNPs were preheated to 50° C. and stirred at 550 rpm. For every 10 ml of CPSNP, 1 ml of EDC (2 mg/ml) was added drop-wise to the particle suspension. After 5 min, 1 ml of Sulfo-NHS (15 mg/ml) and 1 ml of the 2 kDa mPEG-amine tether (6 mg/ml) were added drop-wise. The reaction proceeded for 15 h. Excess EDC, Sulfo-NHS, and mPEG were removed from the retentate with the 30 kDa Amicon Centrifugal Filter at 5000 g (6755 rpm) for 2-3 min. The filtrate was filtered once more to maximize particle recovery.

The presence of mPEG on the surface of CPSNPs was verified with the Brookhaven Instruments zeta potential analyzer in ZetaPlus software v. 3.23 (Holtsville, N.Y.). Suspensions were diluted 1:1 to 1:5 in 70/30 ethanol/water (pH ˜7.4-9.0). Solvent parameters were 1.363 for the refractive index, 2.025 cP for viscosity, and 30.23 for dielectric constant. Five runs were averaged per sample as listed in Table 1. These values were combined into FIG. 1c with 95% confidence intervals. Particles were imaged on the FEI Tecnai G² Spirit BioTWIN TEM (Materials Characterization Lab, Pennsylvania State University) at 120 kV. Selected CPSNP samples were diluted 1:3 in 70/30 ethanol/water (pH ˜7.4-9.0) and a drop was transferred onto a copper grid (CF-300Cu, Electron Microscopy Sciences). The images were processed and quantified with Image J (NIH) and the data was transferred to Origin (OriginLab) for lognormal fitting (n=1300-1900 particles).

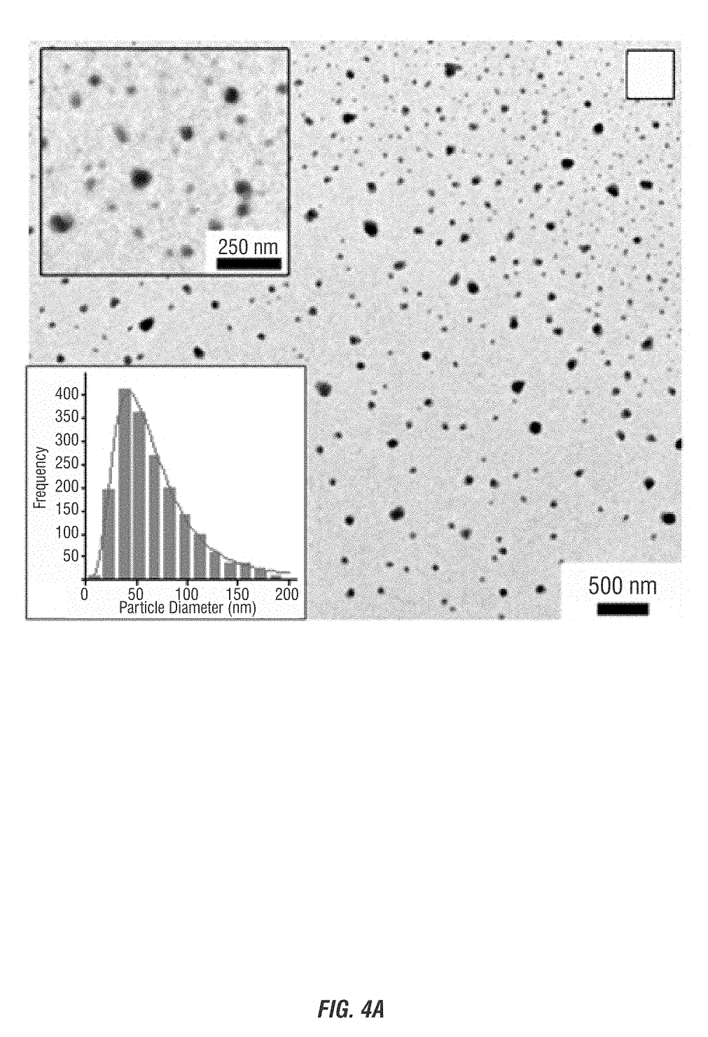

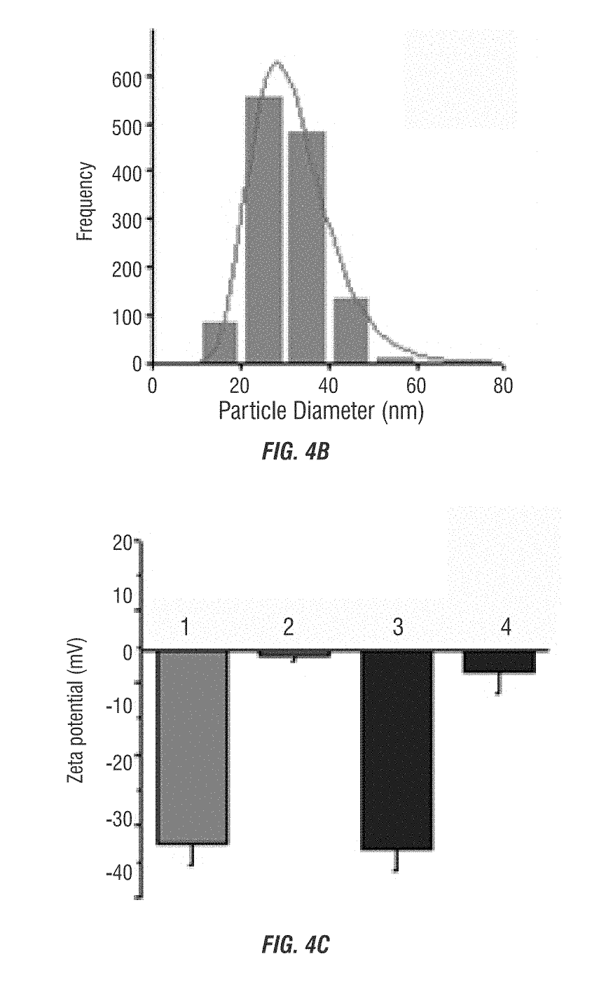

The CPSNP diameters, which are shown in FIGS. 4A and B to be less than 100 nm, lie within the range shown to be optimal for penetration into pancreatic tumors.⁴⁸ A lack of efficacy in conventional pancreatic cancer treatment has been attributed in part to poor drug delivery to tumor cells, since PDAC tumors are not well vascularized with extensive desmoplastic stroma.¹⁷

The synthetic preparation of mPEG-terminated CPSNPs (FIG. 1) used a reverse micelle approach (11-14, 16, 32, 35, 36) and resulted in CPSNPs with a mean diameter of <100 nm; a range that is optimal for nanoparticle penetration into fibrotic pancreatic tumors (16, 37). The mean particle size distribution (±□_{z}, the lognormal standard deviation) of the mPEG-CPSNPs was 30 nm (±□_{z}=0.28) (FIG. 2B) and the distribution for mPEG-FdUMP-CPSNPs was 57 nm (±□_{z}=0.55) (FIG. 2A). CPSNPs were surface functionalized with citrate and methoxy-terminated PEG to improve colloidal stability (36). Zeta potential was measured to monitor changes in surface functionalization (FIG. 2C). The average zeta potentials for Cit-CPSNPs (C1) and Cit-FdUMP-CPSNPs (C2) were −3 5±4 mV and −36±4 mV, respectively. PEGylation leaves a methoxy group termination on the CPSNP surface, resulting in a near neutral zeta potential for mPEG-CPSNPs (C3) and mPEG-FdUMP-CPSNPs (C4) of −1±1 mV and −5±4 mV, respectively.

A preliminary investigation with the FEI Titan³ TEM (Materials Characterization Lab, Pennsylvania State University) on the encapsulation mechanism of CPSNPs revealed that the nanoparticles are composed of ˜1-2 nm sub-particles. The hypothesis is that upon inter-micellar exchange of the calcium and phosphate precursors, these sub-particles are immediately produced. Drug molecules adsorb to the surfaces and become encapsulated as the sub-particles agglomerate into the final 15-60 nm CPSNP during secondary nucleation and growth. This agglomerative-growth process is well-known in the Stöber silica system^{49, 50, 51} and is speculated to be observed for the first time in the Ca—P—H₂O and Ca—P—Si—H₂O systems. Because CPSNPs are composed mainly of low Z-number, or low contrast, elements, the material is subjected to beam damage without cryo-EM and surface features are indistinguishable. Thus, a high Z-number element, osmium, was incorporated as osmium (III) chloride to partially substitute calcium chloride. Bright field TEM showed ˜1-2 nm osmium-containing particles in 60 nm CPSNPs. Dark field HAADF imaging suggests that these osmium sub-particles are homogenously distributed throughout the particle. Elemental mapping suggests that the expected elements, calcium, oxygen, osmium, and phosphate are present.

Colloidal stability of mPEG-X-CPSNPs is maintained by the steric repulsion of the polymer coated surface, which prevents agglomeration and enables the delivery of the nanoparticles in physiological conditions. The PEGylation procedure is a widely used click chemistry reaction, which in this case, involves the crosslinkage of carboxyl groups and primary amines on mPEG in the presence of EDC and Sulfo-NHS.^{52,53} In addition, it is essential to buffer the CPSNP suspension with KOH for long-term storage after PEGylation to prevent significant pH drop when acidic mPEG, EDC, and Sulfo-NHS precursors are introduced. Dissolution of calcium phosphate is known to occur at pH<7.0.

The averaged zeta potentials in FIG. 4 confirms the success of PEGylation from Cit-X-CPSNP to mPEG-X-CPSNP. The reduced magnitude in zeta potential between C1-C2 and C3-C4 is the result the change of surface functionalization. Exposed carboxyl groups from citrate are crosslinked to the primary amines on one end of mPEG, which leaves a neutrally charged methoxy group termination on the surface of CPSNPs. Examples of this surface charge shift was also previously demonstrated by the Adair group.

### Example 3

### In Vitro Evaluation of Free Drug and CPSNP Encapsulated FdUMP

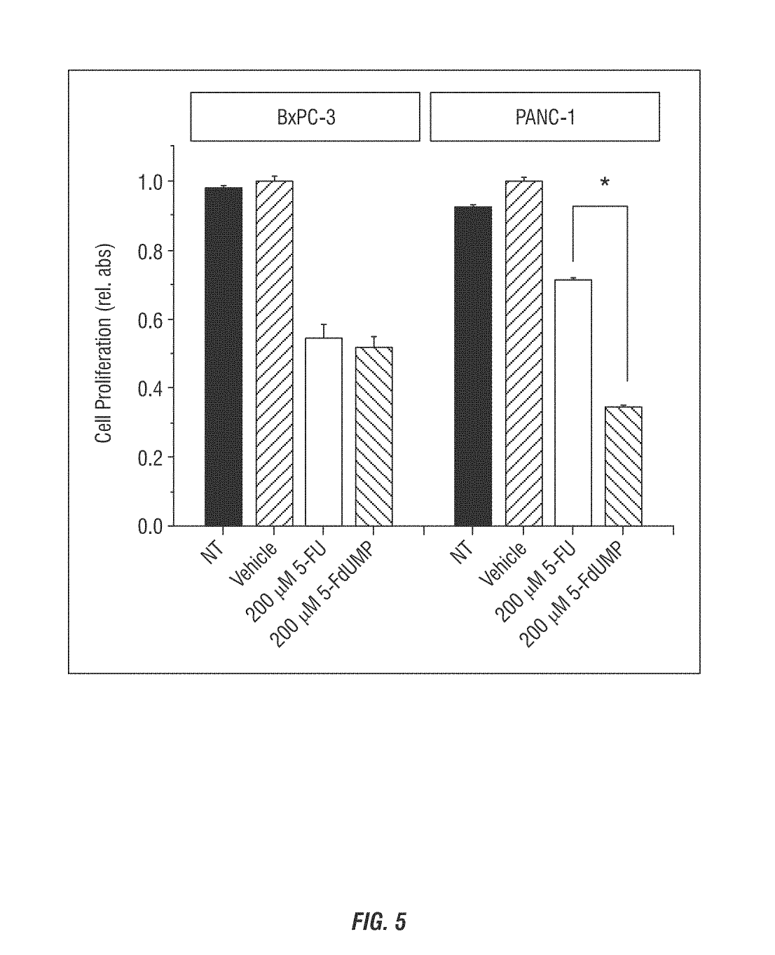

The use of nanocarriers to deliver chemotherapeutic drugs can reduce toxic side-effects, increase efficacy, and obviate the drawbacks of poorly soluble drugs. These studies investigated whether CPSNPs can encapsulate FdUMP and deliver the active drug to cancer cells at an adequate dose to block tumor cell proliferation. While others have reported that polymeric FdUMP has efficacy against other cancers,⁴² the effect of FdUMP on pancreatic cancer cells, which are more inherently resistant to chemotherapeutic drugs, had not been shown. Initial studies therefore focused on the effect of free 5FU and FdUMP on the proliferation of the cultured human pancreatic cancer cell lines, Bx-PC3 and PANC-1 as shown in FIG. 5. BxPC-3 has been shown to be more sensitive to 5FU and gemcitabine, while PANC-1 is more resistant to both drugs.^{54,55} Referring to FIG. 5, in vitro proliferation of human pancreatic cancer cell lines BxPC-3 and PANC-1 after no treatment (NT), treatments with PBS vehicle, and 200 μM of free 5FU and FdUMP. While both 5FU and FdUMP were equally effective in decreasing BxPC-3 proliferation, the equivalent dose of FdUMP was twice as effective as 5FU in reducing PANC-1 proliferation, *p=0.01.At a dose equivalent to the IC₅₀ of 5FU, 200 uM free FdUMP was twice as effective at reducing PANC-1 proliferation as was free 5FU, but equally effective as 5FU for BxPC-3 cells. The concentration of free FdUMP required for PDAC cell growth inhibition is much higher than that demonstrated for the FdUMP polymer F10 against the glioblastoma cell line G48a (IC₅₀ of 1 uM),³⁹ or prostate cancer cells PC3 and C4-2 cells (IC₅₀ values for F10 in the nanomolar range).⁴² This could in part be due to the difference in drug formulation−prior studies comparing equal amounts of free FdUMP and the F10 polymer have shown that F10 is more efficacious than the equivalent dose of FdUMP.³⁵ 5 FU is known to be transported into the cell by nucleoside transporter systems, including human equilibrative nucleoside transporters (hENTs), which do not transport nucleotides such as FdUMP. However, the inherent chemoresistance of PDAC cells likely also to play a role in drug efficacy.

### Example 4

### Efficacy of Nanoparticle Encapsulation

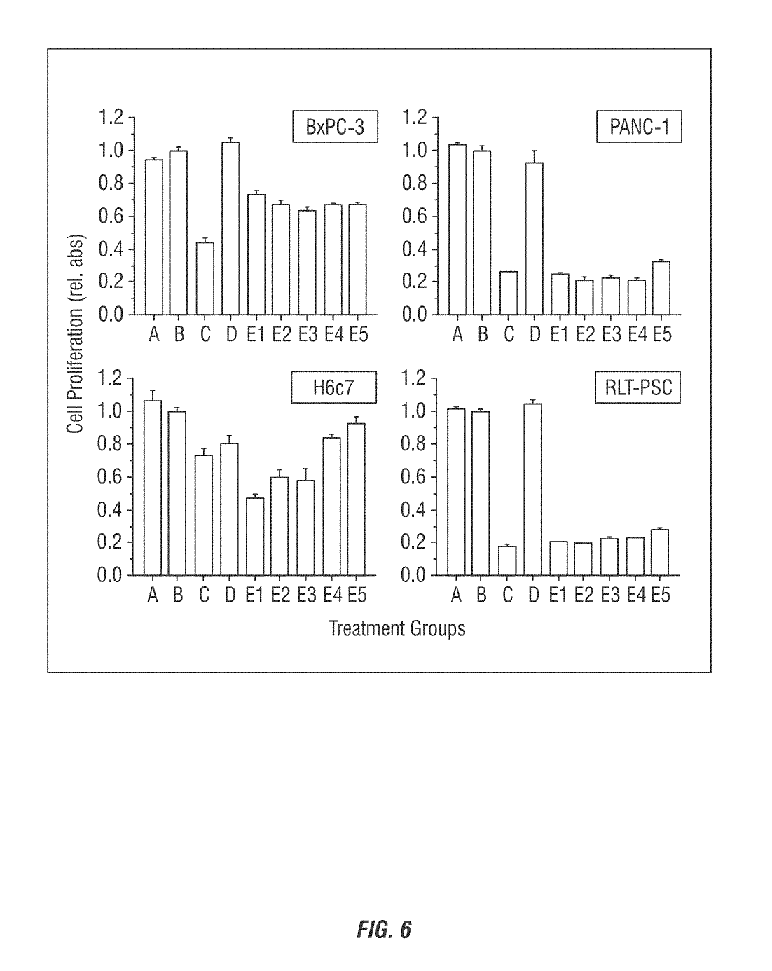

To test whether nanoparticle encapsulation would improve FdUMP efficacy, free FdUMP and mPEG-FdUMP-CPSNPs were used to treat BxPC-3, H6c7, PANC-1, and RLT-PSC cells in FIG. 6 A-D. A greater pancreatic cancer cell knock-down effect by mPEG-FdUMP-CPSNPs than the free, unencapsulated drug was observed. The in vitro proliferation of human pancreatic cancer cell lines BxPC-3 and PANC-1, human pancreatic ductal epithelial cell line H6c7, and human pancreatic stellate cell line RLT-PSC was measured at 72 hours after (A) no treatment, (B) PBS vehicle, treatment with (C) 200 μM free FdUMP, (D) mPEG-Ghost-CPSNPs, and mPEG-FdUMP-CPSNPs in decreasing doses, (E1) 21 μM, (E2) 840 nM, (E3) 420 nM, (E4) 280 nM, and (E5) 210 nM. MPEG-FdUMP-CPSNPs blocked the growth of pancreatic cancer cells and stellate cells while having a lesser effect on normal human pancreatic ductal cell proliferation. Proliferation was normalized to the vehicle control for each cell line.

CPSNPs in 70/30 ethanol/water suspensions were completely dried under pre-purified argon and reconstituted in sterile 1×PBS without calcium or magnesium. MPEG-FdUMP-CPSNPs were added to cells at the equivalent doses of 21 μM, 840 nM, 420 nM, 280 nM, and 210 nM, along with 200 μM free FdUMP as a positive control.

BxPC-3 and Panc-1 were obtained from ATCC. RLT-PSC pancreatic stellate cells were a gift from Professor Ralf Jesenofsky, University of Heidelberg, and H6c7, immortalized human pancreatic ductal epithelial cells, were a gift of Dr. Ming-Sound Tsao, University of Toronto. H6c7 cells have a near normal genotype with wild type p53 and KRAS genes. Cells were cultured in the appropriate media as follows: Dulbecco's modified Eagle medium with 10% FBS for PANC-1 and RLT-PSC, RPMI 1640 with 10% FBS for BxPC-3, and complete keratinocyte basal medium (KBM), containing growth factors, hormones and bovine pituitary extract for H6c7 cells (Invitrogen).

PANC-1, BxPC-3, RLT-PSC and H6c7 cells were seeded into 96 well plates at 5,000 cells per well. At 24 hours post seeding, media only/no treatment (NT), vehicle (lx sterile PBS without calcium and magnesium), 5FU and FdUMP treatments were initiated and viable cells determinations were made at 24, 48 or 72 hr post-treatment using an alamarBlue® assay (Life Technologies). Data were normalized to vehicle treatments for all time points.

Untreated cells, cells treated with equal amounts of mPEG-Ghost-CPSNPs, and a diluent control, PBS, served as negative controls. No difference in proliferation of untreated cells, PBS-treated cells and cells treated with mPEG-Ghost-CPSNPs was observed, indicating that the CPSNPs alone were non-toxic. PANC-1 and RLT-PSC proliferation was significantly reduced by mPEG-FdUMP-CPSNPs. For both cell types, the lowest dose of mPEG-FdUMP-CPSNPs at 210 nM provided similar cell growth inhibition compared to 200 free FdUMP. A second pancreatic cancer cell line, BxPC-3 was slightly less sensitive to the same concentration of free FdUMP and to mPEG-FdUMP-CPSNPs, although treatment with drug-doped CPSNPs did significantly reduce cell proliferation compared to vehicle-treated control cells. This is consistent with reports that BxPC-3 is more resistant to 5FU than is PANC-1 and contains higher levels of TS mRNA.⁵⁶ Thus by encapsulating FdUMP in CPSNPs, similar efficacy in blocking cellular proliferation was demonstrated using ˜1000× less drug. Unlike PDAC cells or stellate cells, proliferation of the normal pancreatic ductal epithelial H6c7 cells was less affected by the drug-loaded CPSNPs. One potential explanation for the lack of CPSNP effect on these non-transformed (wild-type Kras) cells could be the route of nanoparticle uptake. Unlike normal pancreatic ductal cells, pancreatic cancer cells with oncogenic Kras have high levels of macropinocytosis, which preferentially internalizes negatively charged macromolecules by enhanced endocytosis.⁵⁷ Others have recently demonstrated that SERRS nanostars, which also are negatively charged, are taken up by PDAC tumor cells, and not normal cells, via this mechanism.⁵⁸

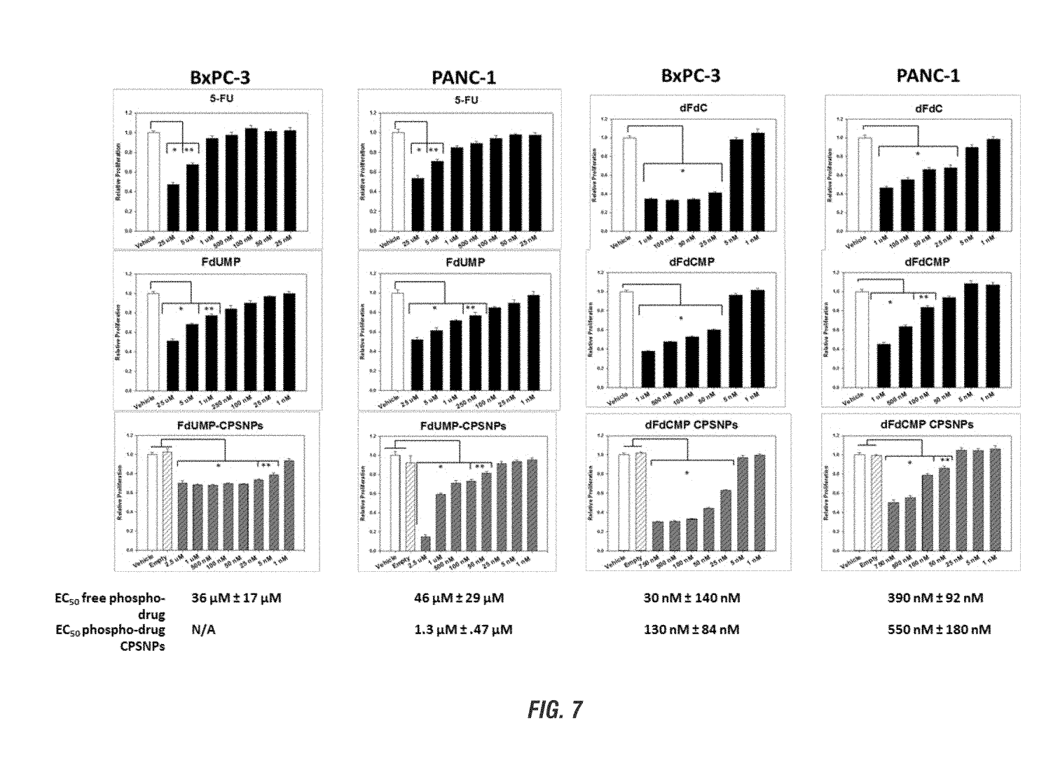

See further FIG. 7. In vitro growth of human PDAC cell lines BxPC-3 and PANC-1 is effectively blocked by mPEG-CPSNPs containing gemcitabine monophosphate (dFdCMP), with EC50 values of 130 and 550 nM, respectively. BxPC-3 cells were more resistant to mPEG-FdUMP-CPSNPS than PANC-1 cells, which had an EC50 of 1.3 □M. Empty CPSNPs (light hatched bars), free drug (dark bars) or drug-containing CPSNPs (dark hatched bars) are expressed as relative proliferation (percent of vehicle controls, white bars). Values are the mean of 3-4 independent experiments with *=p<0.001 and **=p<0.01.

Against both cell lines, 5-FU and FdUMP had similar efficacy, with a low uM EC₅₀ for both compounds and both cell lines (FIG. 4). Similarly, dFdC and dFdCMP were both effective in blocking proliferation of BxPC-3 and PANC-1. EC₅₀ for free dFdCMP against both cell lines was in the nM range and was lower than for 5-FU or FdUMP.

Comparing FdUMP to CPSNP-encapsulated FdUMP, the response of BxPC-3 cells to encapsulated FdUMP plateaued between 2.5 uM and 50 nM, and higher doses failed to affect the proliferation of these cells (FIG. 7). PANC-1 appeared to respond similarly to both free FdUMP and encapsulated FdUMP, with an EC₅₀ for FdUMP-CPSNPs of 1.3 □M. As noted, both BxPC-3 and PANC-1 were more sensitive to dFdCMP-CPSNPs than to FdUMP-CPSNPs. The EC50 for dFdCMP-CPSNPs against PANC-1 was 550 nM, and proliferation of BxPC-3 was decreased to a much greater degree by dFdCMP-CPSNPs (EC₅₀ 130 nM) than by FdUMP-CPSNPs. There was no significant difference between the efficacy free dFdCMP and encapsulated dFdCMP for either cell line. Again, neither BxPC-3 nor PANC-1 proliferation was affected by empty mPEG-CPSNPs, consistent with previous work indicating that the particles themselves were non-toxic. Thus nanoparticle encapsulation of FdUMP and dFdCMP did not affect the ability of these phospho-drugs to inhibit the growth of PDAC cell lines in vitro.

Pancreatic stellate cells (PSCs) also stimulate tumor growth and contribute to the fibrotic microenvironment commonly found in pancreatic tumors. Tumor fibrosis inhibits the penetration of anti-cancer drugs by decreasing tumor vascularity and creating physical barriers. Previously, we and others have shown that CCKA and B receptors are present on pancreatic stellate cells and when stimulated by CCK, PSCs increase stromal collagen synthesis, contributing to tumor fibrosis. Reducing stellate cell growth with targeted nanoparticles could make other treatments more effective.

### Example 5

### Encapsulated FdUMP Inhibits Thymidylate Synthase

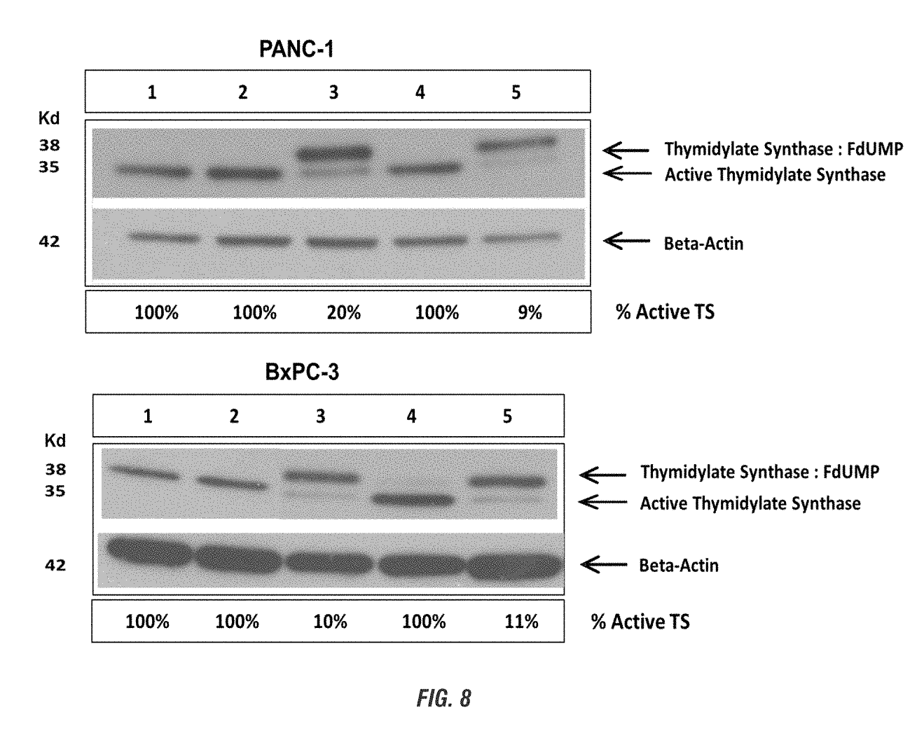

Inhibition of thymidylate synthase by FdUMP occurs because a covalent bond forms between the drug and a cysteine near the TS active site. Thus in the presence of folate, TS dimers and FdUMP create an irreversibly inactivated enzyme:drug complex.⁵⁹ Experimentally, the inhibition of TS activity by FdUMP can be detected by the presence of this stable ternary complex. Since the inactive ternary complex composed of TS, FdUMP, and THF displays a larger molecular weight than active TS,⁶⁰ immunoblots in FIG. 8 were used to establish that CPSNP-encapsulated FdUMP remained active and inhibited thymidylate synthase in treated pancreatic cancer cells.

PANC-1 cells were incubated for 24 hours in the following treatment groups: NT, vehicle, 250 μM free FdUMP, 200 nM FdUMP-CPSNPs, and Ghost-CPSNPs in PBS. Lysates were collected by aspirating the media, washing with 1×PBS, and adding RIPA buffer containing Complete Mini protease cocktail (Roche). Lysates were spun to remove debris and the supernatants frozen at −80° C. Protein concentration was determined by micro BCA protein assay (Thermo Scientific) and 20 μg of protein separated by gel electrophoresis. After transfer to HyBond ECL and blocking for 1 hr in 5% BSA, blots were probed overnight with anti-TS antibody (1:1000, D5B3, #9045 Cell Signaling). Blots were washed, probed with anti-rabbit-HRP secondary antibody for 1 hour and developed with Pierce Pico (Thermo Scientific). Quantitation of scanned blots was done using Image J software.

In the presence of folate, TS dimers and FdUMP create an irreversibly inactivated enzyme:drug complex (59, 68), and the inactive ternary complex (TS, FdUMP and THF) displays a larger molecular weight than active TS on immunoblots (460). Immunoblot analysis of TS in untreated cells, cells treated with vehicle or cells treated with mPEG-CPSNPs did not demonstrate formation of a TS ternary complex and only the lower molecular weight, active form of TS was present in these cells (FIG. 8, Lanes 1, 2, and 4). Treatment with free FdUMP shifted most of the active TS into the inactive ternary complex, as expected, although FdUMP was slightly less effective for PANC-1 cells (80% inhibition) than for BxPC-3 cells (90% inhibition)(FIG. 8, Lane 3). However, nearly all TS was in a catalytically inactive TS:FdUMP:THF ternary complex when BxPC-3 and PANC-1 cells were treated with mPEG-FdUMP-CPSNPs (89%-91% inhibition; FIG. 8, Lane 5). Thus when delivered in vitro, CPSNP-encapsulated FdUMP retained its capacity to bind to and inhibit its target enzyme, TS, in both BxPC-3 and PANC-1 cells.

For PANC-1 cells of BxPC-3 treated with either free FdUMP or with mPEG-FdUMP-CPSNPs nearly all the TS was in a catalytically inactive TS:FdUMP:THF ternary complex (Lanes 3 and 5). See FIG. 8. Immunoblots of the FdUMP target enzyme thymidylate synthase (TS) from PANC-1 (upper panel) or BxPC-3 (lower panel) cells treated with 250 μM free FdUMP (Lane 3) or 2 μM mPEG-FdUMP-CPSNPs (Lane 5). Both cell lines showed significant (>80%) conversion of TS to an inactive ternary complex (TS:FdUMP) with free drug and with mPEG-FdUMP-CPSNP treatment. Controls that received no treatment (Lane 1), PBS vehicle (Lane 2) or mPEG-CPSNPs containing no FdUMP (Lane 4) exhibited only active TS with no evidence of TS:FdUMP ternary complex formation.

Cells that were untreated, treated with PBS vehicle, or with mPEG-Ghost-CPSNPs, did not exhibit TS ternary complex formation, and only the lower molecular weight, active form of TS was present (Lanes 1, 2, and 4). This indicates that free FdUMP inactivated TS as expected and that encapsulated FdUMP did not reduce its efficacy. In fact, nanoparticle delivered FdUMP was more effective at inhibiting TS than free drug. Treatment of PANC-1 cells with mPEG-FdUMP-CPSNPs converted over 90% of TS into the ternary complex form, while free FdUMP treatment resulted in only 80% ternary complex formation.

### Example 6

### Cell Cycle Arrest in FdUMP-Treated PANC-1 Cells

Nuclear DNA content reflects the position of a cell within a cell cycle, and since TS provides the sole de novo source of cellular thymidylate, which is necessary for DNA replication and repair, inhibition of TS and depletion of nucleotide pools will eventually lead to DNA strand breaks and arrest of the cell cycle. The following confirms that the impairment of cell proliferation by mPEG-FdUMP-CPSNPs is consistent with the known mechanism of action for this drug.

For determination of nuclear DNA content, PANC-1 treatments included NT, vehicle (1× sterile calcium- and magnesium-free PBS), 250 μM free FdUMP, Ghost-CPSNPs, or 200 nM FdUMP-CPSNPs. After 72 hours of treatment, cells were fixed in 75% ethanol overnight. Immediately prior to analysis, cells were treated with 1 μg/mL of RNase A and stained with 50 μg/mL of propidium iodide, which is taken up by double-stranded DNA. DNA content was determined using a FACSCalibur flow cytometer (BD Biosciences), and data were analyzed with Cellquest (Verity Software).

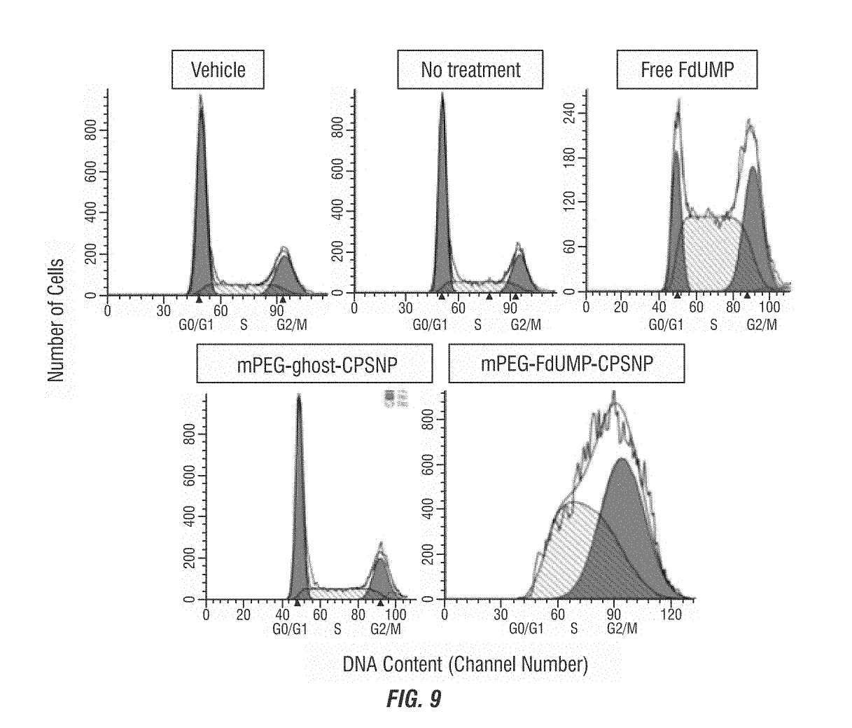

PANC-1 cells treated with these CPSNPs were analyzed for cell cycle progression by determining cellular DNA content as shown in Table 3 and FIG. 9.

TABLE 3
TABLE 3. Cell cycle analysis of PANC-1 after treatment with free
FdUMP and mPEG-FdUMP-CPSNPs.

| | % Cells | | % Cells in |
|---|---|---|---|
| Treatment Group | in G1/G0 | % Cells in S | G2/M |
| No treatment | 54.1 ± 4.0 | 24.6 ± 1.5 | 21.4 ± 1.2 |
| PBS Vehicle | 53.8 ± 4.0 | 23.6 ± 1.3 | 22.7 ± 1.5 |
| mPEG-Ghost-CPSNPs | 52.0 ± 3.8 | 25.1 ± 1.6 | 22.6 ± 1.0 |
| 250 μM free FdUMP | 20.3 ± 3.0 | 56.3 ± 10.3 | 23.5 ± 6.3 |
| 200 nM mPEG-FdUMP- | 0.1 ± 0.03 | 44.8 ± 7.0 | 55.1 ± 5.3 |
| CPSNPs | | | |

To determine the fraction of cell population in each cell cycle phase, the DNA content distribution was deconvolved using Cellquest software. Percentage of the total cells in each cell cycle phase was determined using a non-parametric curve-fitting method for histogram decomposition and are expressed as the average +/− standard error of the mean of three independent experimental replicates.

Untreated cells, vehicle-treated cells, and mPEG-Ghost-CPSNP-treated cells all had equivalent percentages of cells in S-phase, indicating that the nanocarriers themselves had no effect on cell division. Compared to these controls, cells treated with 250 uM of free FdUMP demonstrated a partial S-phase arrest and a reduced number of cells in G1/G0 phase. The percentage of free FdUMP-treated cells in the G1/G0 phase decreased from 54% to 20%, and the percentage of cells in the S phase of the cell cycle increased from 25% to 56% compared to untreated cells. Furthermore, cells treated with 200 nM mPEG-FdUMP-CPSNPs were completely arrested and showed no G1/G0 phase cells. The percent of cells in G2/M and in S phase was nearly equal, indicating a complete block in proliferation. Thus the arrest of cell division in cells treated mPEG-FdUMP-CPSNPs was even more pronounced than in free FdUMP-treated cells, despite the fact that the drug concentration delivered by CPSNPs was significantly lower (200 nM encapsulated drug versus 250 uM free drug). The observed cell cycle arrest of PANC-1 cells by mPEG-FdUMP-CPSNP is consistent with the known mechanism of action for FdUMP, and has been also seen for FdUMP-treated colon cancer cells.⁶¹ This is additional evidence that mPEG-FdUMP-CPSNPs can deliver active drug to cancer cells and induce cell cycle arrest.

### Example 7

### Effects of Encapsulated GemMP on Cancer Cells

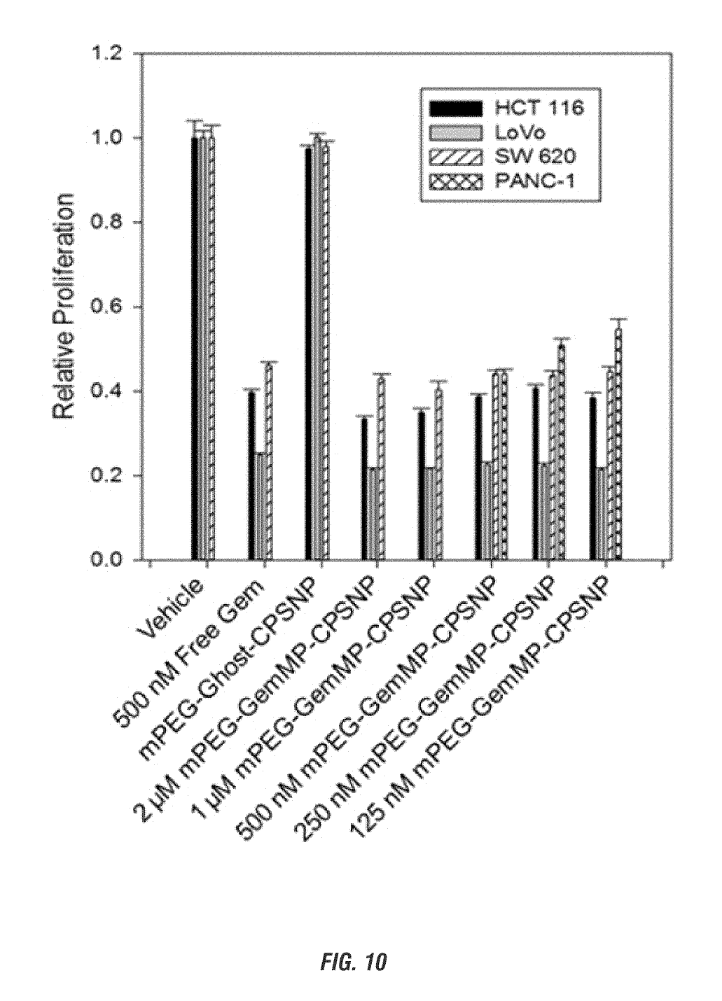

The effects of encapsulated GemMP in mPEG-GemMP-CPSNPs were also assessed in vitro on PANC-1 cells and colon cancer cell lines, LoVo, HCT116, and SW620, in FIG. 10. This initial data shows that GemMP at 125 nM is just as effective as 500 nM of the free non-phosphorylated Gem, which suggests similar efficacy as mPEG-FdUMP-CPSNPs and that CPSNP treatment is versatile for treating other types of cancers. While studies are ongoing, the colon cancers may be similarly or more sensitive to mPEG-GemMP-CPSNPs as PANC-1. Reduced cell proliferation was not observed with the control particles, mPEG-Ghost-CPSNPs, which further support that CPSNPs are made of a non-cytotoxic material.

### Example 8

### CCKBR-Targeted FdUMP-CPSNPS Deliver Active Drug to PDAC Tumor Cells In Vivo.

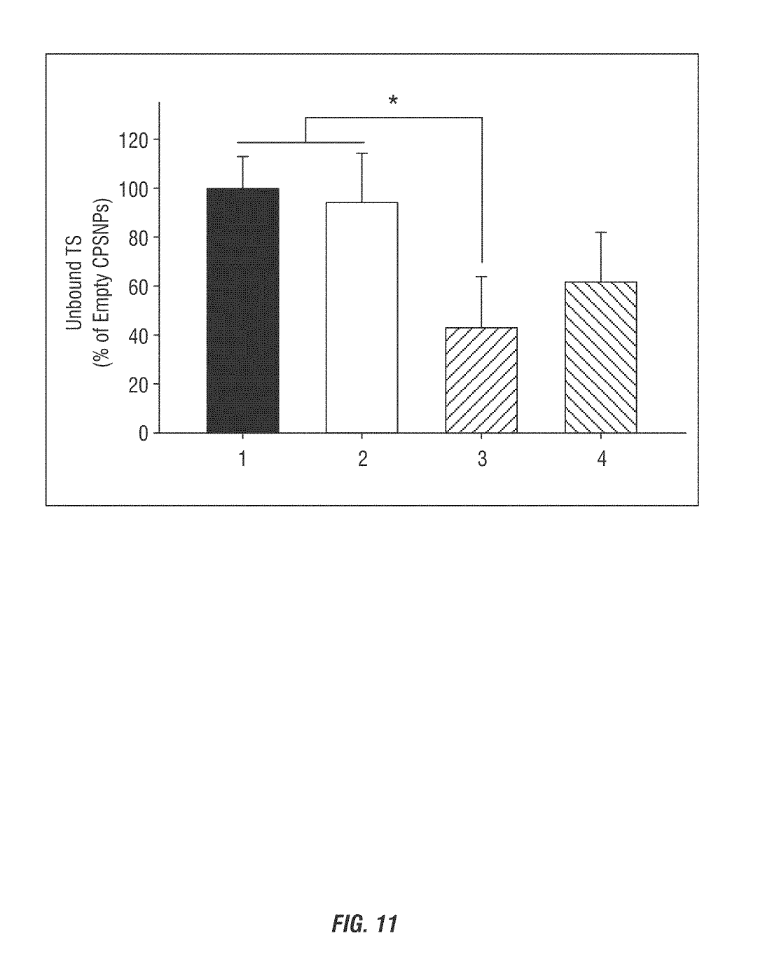

We have identified a DNA aptamer, a small structured oligonucleotide, which can bind to a receptor on the surface of PDAC tumor cells, the CCK-B receptor, and trigger receptor internalization (69). This aptamer is a nucleic acid molecule that selectively binds to a pancreatic adenocarcinoma cell and other cholecystokinin type B receptor (CCKBR) and which recognizes amino acid regions 5-21 and/or 40-57 of a CCKBR such as that found at NCBI Reference Number No._795344.1 (2015). When this aptamer was attached to the surface of fluorescent CPSNPs it significantly enhanced CPSNP accumulation in murine PANC-1 tumors. In this study, either CCKBR aptamer or the endogenous CCKBR peptide ligand gastrin, were attached to mPEG-FdUMP-CPSNPS using established protocols (8). The ability of these targeted nanoparticles to deliver active FdUMP to PDAC tumor cells in vivo was assessed by TS immunoblotting of tumor tissues. Active TS protein levels in tumors from mice treated with empty-CPSNPs and untargeted-FdUMP-CPSNPS were not significantly different from each other (FIG. 11). This indicated that CPSNPs without tumor-specific targeting are poorly taken up by PDAC tumors in vivo. Tumors from mice treated with FdUMP-CPSNPs surface bioconjugated with gastrin-16 peptide, which also binds to CCKBR on PDAC tumor cells, had reduced TS protein levels compared to untargeted-FdUMP-CPSNPs. This is consistent with previous studies which showed that fluorescent CPSNPS targeted with gastrin had enhanced PDAC tumor uptake in vivo (8). Tumors from mice treated with aptamer targeted-FdUMP-CPSNPs had the lowest active TS levels of all treatment groups—a 60% reduction versus both empty CPSNPS and untargeted FdUMP-CPSNP controls, *p<0.05. This suggests that significantly more FdUMP cargo was internalized by PDAC tumors from mice treated with aptamer-FdUMP-CPSNPs. This result is again consistent with recent data showing that this CCKBR aptamer enhanced fluorescent CPSNP accumulation in PANC-1 tumors in vivo compared to untargeted or gastrin-targeted particles (69). However, the previous studies with aptamer-targeted fluorescent-CPSNPs did not specifically address cellular internalization of these nanoparticles, as the fluorescent CPSNPS could have been on the tumor cell surface (i.e. associated with CCKBR at the plasma membrane) rather than inside of the tumor cells. These studies clearly demonstrate that targeted-FdUMP-CPSNPs were internalized by PDAC tumor cells. Intracellular accumulation of the TS inhibitor FdUMP by PDAC tumor cells in vivo was also most effective when the FdUMP was delivered by aptamer-targeted mPEG-FdUMP-CPSNPs.

These results indicate that encapsulated FdUMP is an effective anti-proliferative agent for pancreatic cancer cells and pancreatic stellate cells. Due to ionic interactions between phosphate groups on the drug molecule and calcium in the nanoparticles, FdUMP was encapsulated in CPSNPs to a significantly greater degree than either 5FU or FUdR. This suggests that other phosphorylated compounds can also be effectively encapsulated into CPSNPs, opening up the potential for using CPSNPs as a vehicle to deliver other chemotherapeutic drugs. For example, a recent meta-analysis suggested that although PDAC patients may benefit from 5FU-gemcitabine combination therapy, significant toxicities, including neutropenia, thrombocytopenia and diarrhea, can occur. Since this is in part due to the high drug concentrations required to achieve a therapeutic response, a treatment strategy which combines CPSNPs that encapsulate FdUMP with CPSNPs containing an active metabolite of gemcitabine could increase the anti-tumor efficacy of the drug combination with lower toxicity. If combined with agents that normalize tumor vasculature, it is possible that efficacy could be increased even further⁶³.

In addition to encapsulating effective concentrations of FdUMP, CPSNPs delivered the FdUMP to cells in a biologically active form. Evidence that encapsulated FdUMP retained activity was the formation of thymidylate synthase ternary complexes and complete cell cycle arrest in treated pancreatic cancer cells. The concentration of encapsulated FdUMP needed to block cancer cell growth was more than 1000 times less than the amount of free FdUMP required to achieve a similar effect. This suggests that CPSNPs are effective drug delivery vehicles. The potential for encapsulated FdUMP to also impair pancreatic stellate cell function within the tumor microenvironment is significant. Because the tumor microenvironment is a major barrier for the effective delivery of therapeutics, any treatment that can reverse stromal fibrosis by reducing the number of activated stellate cells is likely to have a synergistic effect with traditional chemotherapeutics⁶⁴⁻⁶⁵ and could reduce metastases.⁶⁶ However since two recent studies suggest that a complete ablation of cells within the pancreatic tumor stroma can result in more aggressive tumor cell growth⁶⁷⁻⁶⁸ a balanced approach to targeting tumor versus stromal cells should be taken.⁶⁹

CPSNPs serve as a novel vehicle which can effectively encapsulate phosphorylated compounds such as FdUMP which, by definition, are limited by poor bioavailability and/or poor cellular internalization. During systemic administration, the plasma concentration of chemotherapeutic drugs can increase rapidly then quickly drop as the drug is metabolized. By encapsulating chemotherapeutics into nanoparticles which are stable in circulation, a more uniform drug delivery can be achieved—reducing the chance of both under-dosing and overdosing. If drug-containing CPSNPs can be surface modified to direct these particles selectively to cancer cells, their overall efficacy could be enhanced even further. This study demonstrated a promising new methodology for encapsulating and delivering chemotherapeutic agents to pancreatic cancer cells and supports current developments in targeting pancreatic cancer in vivo.

A functional group on a drug and/or imaging agent that results in a covalent bond with Ca2+ making up a calcium ligand binding species defined by [Ca:L]2-z species that is strongly incorporated as part of a resorbable, amorphous calcium phosphate or calcium phosphosilicate material. Various embodiments provide for encapsulation of phosphorylated drugs or imaging agents, often metabolic products of the prodrug, within nanoparticles; where the nanoparticle is selected from a group of nanoparticle materials including resorbable amorphous materials; where the amorphous resorbable nanoparticle is calcium phosphosilicate or calcium phosphate; where the phosphorylated metabolic drugs are chemotherapeutics; where the phosphorylated chemotherapeutics are selected from anti-cancer drugs; where the anti-cancer drugs are anti-metabolites, micro-tubule disruptors, platin-based drugs, and the like; where the anti-metabolities are 5-fluorouracil and gemcitabine; where the 5FU and phosphorylated gemcitabine are encapsulated at greater than 10⁻⁵M in calcium phosphosilicate nanoparticles ranging in diameter from 10 nm to 200 nm; where the preferred embodiment is 10⁻⁵M FdUMP encapsulated in the CPSNPs; where the preferred embodiment is 10⁻³M FdUMP encapsulated in the CPSNs; where the preferred embodiment of the FdUMP encapsulated in the CPSNPs have a diameter from 30 to 70 nm; where the nanoparticle surface is conjugated with a PEG chosen from a PEG with terminal groups ranging from hydroxy, methoxy, malemidie, amine, carboxy, etc functional groups; where the preferred embodiment is MPEG; where the preferred embodiment is a PEG carboxy, maleimide, amine, etc. terminal group coupled with a carbodiimide-sulfo NHS strategy to a polypeptide or protein or other small molecule; and where the formulation pH is maintained at pH 7.0 to pH 7.5 once the bioconjugated and redispersed surface functionalized phosphorylated drug metabolite encapsulated CPSNP is suspended in phosphated buffered saline.

### Materials and Methods

### CPSNP Reagents and Materials.

The HPLC stationary phase consisted of solid glass microspheres ˜200 μM in diameter (Spheriglass A-Glass 1922, Potters Industries) that was soaked in purified water for 48 h and thoroughly rinsed with 10⁻³M HCl (Sigma-Aldrich) and 10⁻³M NaOH (J. T. Baker) solutions before use. The stationary phase was wet-packed in a 5 cm long×⅜″ OD, ¼″ ID polycarbonate tube (McMaster-Carr). General chemicals include Igepal CO-520 (Rhodia Chemical Co.), cyclohexane (Alfa Aesar), 5-fluorouracil (5FU, ≥99%, Sigma-Aldrich), 5-fluoro-2′-deoxyurdine (FUdR, ≥98%, Tocris), 5-fluoro-2′-deoxyuridine-5′-monophosphate (FdUMP, ˜85-91%, Sigma-Aldrich), adenosine 5′-triphosphate disodium salt hydrate (ATP, 99%, Sigma-Aldrich), gemcitabine hydrochloride (Gem, ≥99%, Sigma-Aldrich), gemcitabine monophosphate formate salt (GemMP, 95%, TRC Canada) calcium chloride dihydrate (CaCl₂, ≥99%, Sigma-Aldrich), sodium hydrogen phosphate (Na₂HPO₄, ≥99%, Sigma-Aldrich), sodium metasilicate (Na₂SiO₃, Sigma-Aldrich), sodium citrate dihydrate (Cit, ≥99%, Sigma-Aldrich), potassium hydroxide pellets (KOH, J. T. Baker) for pH adjustment, neat ethanol (Koptec). N-(3-dimethylaminopropyl)-N′-ethylcarbodiimide hydrochloride (EDC) was purchased from Sigma-Aldrich and N-hydrosulfosuccimide (Sulfo-NHS) was from Thermo Scientific Pierce. The 2 kD methoxy-polyethylene glycol-amine tether (mPEG) was purchased from JenKem Technology. Reagents were all used without further purification. Aqueous solutions were filtered with 0.2 μm cellulose acetate syringe filters (VWR) before use. Deionized-distilled water was obtained from a High-Q 200PT-SYS (RO/IX2) purification system and purged with pre-purified argon. Purified water was tested for endotoxins (<0.100 EU/mL) using a Charles River Limulus Amebocyte Lysate Endochrome kit.

### CPSNP Synthesis and Laundering.

CPSNPs were prepared by a reverse-micelle water-in-oil (Igepal CO-520/cyclohexane/water) microemulsion method and laundered by van der Waals HPLC. In a standard formulation, two microemulsions with 650 μl of 10⁻² M CaCl₂.H₂O_{(aq)} in 14.06 ml of 29 vol % Igepal CO-520/cyclohexane and 65 ul of 6.0×10⁻² M Na₂HPO_{4(aq)}/65 ul of 8.2×10⁻³M Na₂SiO_{3(aq)}/520 μl of purified water in 14.06 ml of 29 vol % Igepal CO-520/cyclohexane were equilibrated for 15 min with constant stirring at −200 rpm. For drug-doped CPSNPs, the 520 μl of purified water used for Ghost-CPSNPs was substituted by an equal volume of drug agent to maintain the micelle aqueous pool size, ω=4. The starting concentration of the 5FU precursor was 6.3×10⁻³ M, 0.1 M for 5FU:ATP, 9.2×10⁻³M for FdUMP, and 2.5×10⁻³ M for Gem and GemMP. Micellar exchange occurred for 2 min and the reaction was quenched with 225 μl of 10⁻² M citrate for 15 min. The CPSNPs were released from the micelles by the addition of 50 ml of ethanol (pH ˜7.4-9.0). The suspension was loaded in aliquots onto the HPLC column at a flow rate of 2.5-3 ml/min for 10 min and was monitored by UV-Vis absorption (λₘₐₓ=269-276 nm, set to the wavelength of drug agent or Igepal CO-520). The mobile phases were pH adjusted to pH 7.4-9.0 with KOH to mitigate CPSNP dissolution. Excess surfactant and reagents were laundered from the column for 20-30 min with neat ethanol until the absorption baseline was reached and CPSNP fractions were collected in 70/30 ethanol/water (v/v) for 1 min after elution. The load, launder, and collection cycles were repeated until the entire suspension was fully laundered twice.

### PEGylation of CPSNPs.

Filtered citrate-functionalized CPSNPs were preheated to 50° C. and stirred at 550 rpm. For every 20 ml of CPSNP, 2 ml of EDC (2 mg/ml) was added drop-wise to the particle suspension. After 5 min, 2 ml of Sulfo-NHS (15 mg/ml) and 2 ml of the 2 kDa mPEG-amine tether (6 mg/ml) were added drop-wise. The reaction proceeded for 15 h. Excess EDC, Sulfo-NHS, and mPEG were removed from the retentate with the 30 kDa Amicon Centrifugal Filter at 5000 g (6755 rpm) for 2-3 min. The filtrate was filtered once more to maximize particle recovery.

### Particle Characterization.

The presence of mPEG on the surface of CPSNPs was verified with the Brookhaven Instruments zeta potential analyzer in ZetaPlus software v. 3.23 (Holtsville, N.Y.). Suspensions were diluted 1:1 to 1:5 in 70/30 ethanol/water (pH ˜7.4-9.0). Solvent parameters were 1.363 for the refractive index, 2.025 cP for viscosity, and 30.23 for dielectric constant. Measurements were averaged from five replicated formulations with 95% confidence intervals. Particles were imaged on the FEI Tecnai G² Spirit BioTWIN TEM (Materials Characterization Lab, Pennsylvania State University) at 120 kV. Selected CPSNP samples were diluted 1:3 in 70/30 ethanol/water (pH ˜7.4-9.0) and a drop was transferred onto a copper grid (CF-300Cu, Electron Microscopy Sciences). The images were processed and quantified with Image J (NIH) and the data was transferred to Origin (OriginLab) for lognormal fitting (n=1300-1900 particles).

### Liquid Chromatography-Mass Spectrometry (LC-MS/MS).

Particles were diluted in 10% methanol with 0.1% formic acid and 5-CU was spiked in as an internal standard. Chromatography was done on a 2.1 mm×10 cm HSS T3 or C18 CSH column (Waters) on a Waters I-class FTN chromatography system with the column temperature at 40° C. Mobile phase A was water with 5 mM ammonium acetate and B was methanol. The flow rate was 0.5 mL/min and the chromatography consisted of holding at 7.5% B for 1 min, increasing to 95% B over 0.5 min, holding at 95% B for 0.5 min before equilibration to starting conditions. Eluate was analyzed by an inline Waters TQ-S mass spectrometer. The capillary was set at 1.0 kV, source temperature at 150° C., desolvation temperature at 600° C., cone gas at 150 L/hr, and desolvation gas flow at 1200 L/hr. Multiple reaction monitoring (MRM) was used to detect 5-CU and FdUMP. The MRM transition used for 5-CU was 145 >42 with the collision energy set at 12. The MRM transition used for FdUMP was 325 >195 with the collision energy set at 12. FdUMP concentrations were determined using TargetLynx version 4.1 (Waters) using an external calibration curve with 1/x weighting.

### Cultured Human Pancreatic Cell Lines.

BxPC-3 and Panc-1 were obtained from ATCC. RLT-PSC pancreatic stellate cells were a gift from Professor Ralf Jesenofsky, University of Heidelberg, and H6c7, immortalized human pancreatic ductal epithelial cells, were a gift of Dr. Ming-Sound Tsao, University of Toronto. H6c7 cells have a near normal genotype with wild type p53 and KRAS genes. Cells were cultured in the appropriate media as follows: Dulbecco's modified Eagle medium with 10% FBS for PANC-1 and RLT-PSC, RPMI 1640 with 10% FBS for BxPC-3, and complete keratinocyte basal medium (KBM), containing growth factors, hormones and bovine pituitary extract for H6c7 cells (Invitrogen).

### Cell Proliferation Assay.

CPSNPs in 70/30 ethanol/water suspensions were completely dried under pre-purified argon and reconstituted in sterile 1×PBS without calcium or magnesium. PANC-1, BxPC-3, RLT-PSC and H6c7 cells were seeded into 96 well plates at 5,000 cells per well. At 24 hours post seeding, media only/no treatment (NT), vehicle (1× sterile PBS without calcium and magnesium), 5FU and FdUMP treatments were initiated and viable cells determinations were made at 24, 48 or 72 hr post-treatment using an alamarBlue® assay (Life Technologies). Data were normalized to vehicle treatments for all time points.

### Thymidylate Synthase Immunoblotting.

PANC-1 cells were incubated for 24 hours in the following treatment groups: NT, vehicle, 250 μM free FdUMP, 200 nM FdUMP-CPSNPs, and ghost-CPSNPs in PBS. Lysates were collected by aspirating the media, washing with 1× PBS, and adding RIPA buffer containing Complete Mini protease cocktail (Roche). Lysates were spun to remove debris and the supernatants frozen at −80° C. Protein concentration was determined by micro BCA protein assay (Thermo Scientific) and 20 □g of protein separated by gel electrophoresis. After transfer to HyBond ECL and blocking for 1 hr in 5% BSA, blots were probed overnight with anti-TS antibody (1:1000, D5B3, #9045 Cell Signaling). Blots were washed, probed with anti-rabbit-HRP secondary antibody for 1 hour and developed with Pierce Pico (Thermo Scientific). Quantitation of scanned blots was done using Image J software.

### Cell Cycle Analysis.

For determination of nuclear DNA content, PANC-1 treatments included NT, vehicle (1× sterile calcium- and magnesium-free PBS), 250 μM free FdUMP, ghost-CPSNPs, or 200 nM FdUMP-CPSNPs. After 72 hours of treatment, cells were fixed in 75% ethanol overnight. Immediately prior to analysis, cells were treated with 1 μg/mL of RNase A and stained with 50 □g/mL of propidium iodide, which is taken up by double-stranded DNA. DNA content was determined using a FACSCalibur flow cytometer (BD Biosciences), and data were analyzed with Cellquest (Verity Software).

### In Vivo Assessment of FdUMP-CPSNP Up Take by PDAC Tumor Cells.

All animal protocols were approved by the Penn State Hershey IACUC committee. Orthotopic PANC-1 tumors were established in male, athymic (nu/nu) mice (Charles River). Pancreata were injected with 5×10⁶ cells and treatment with CSPNPs was initiated at one week post-surgery. Treatment groups (n=4-5 mice per group, with two experimental replicates) included empty (no FdUMP) mPEG-CPSNPs, mPEG-FdUMP-CPSNPS without the addition of targeting agents to the nanoparticle surface, mPEG-FdUMP-CPSNPs surface bioconjugated with gastrin 16 peptide, and mPEG-FdUMP-CPSNPs surface bioconjugation with the CCKBR aptamer AP1153 (34). CPSNPs were administered at a FdUMP dose of 100 □g/kg, or an equal volume of empty CPSNPs, twice weekly via tail vein injection. After six weeks of treatment, tumor proteins were extracted and thymidylate synthase immunoblots were performed as described above.

### Statistical Analysis.

Results were expressed as means±standard error. Student t-tests were used to evaluate statistical significance with a p<0.05 considered to be significant. To calculate EC50±95% CI, nonlinear regression analysis was performed to generate the curve of best fit for the data according to a Sigmoidal regression using a 4-parameter logistic curve calibration [Y=Yo+(a/(1+((X/Xo)\hat{ }b))] in SigmaPlot 12 (Systat, Inc.).

### Abbreviations

CPSNP, calcium phosphosilicate nanoparticle; mPEG, methoxy-terminated polyethylene glycol, Cit, citrate; NT, no treatment; GEM, gemcitabine; GemMP, gemcitabine monophosphate; TS, thymidylate synthase; THF, 5,10-methylene-tetrahydrofolate; FdUMP, 5-fluoro-2′-deoxyuridine 5′-monophosphate; 5FU, 5-fluorouracil; FUdR, 5-fluoro-2′-deoxyuridine; PSC, pancreatic stellate cells.

### REFERENCES

1. Wang, J.; White, W. B.; Adair, J. H. Dispersion of SiO₂-based nanocomposites with high performance liquid chromatography. The journal of physical chemistry. B. 2006, 110 (10), 4679-85.
2. Morgan, T. T.; Muddana, H. S.; Altinoglu, E. I.; Rouse, S. M.; Tabakovic, A.; Tabouillot, T.; Russin, T. J.; Shanmugavelandy, S. S.; Butler, P. J.; Eklund, P. C.; Yun, J. K.; Kester, M.; Adair, J. H. Encapsulation of organic molecules in calcium phosphate nanocomposite particles for intracellular imaging and drug delivery. Nano letters. 2008, 8 (12), 4108-15.
3. Altinoglu, E. I.; Russin, T. J.; Kaiser, J. M.; Barth, B. M.; Eklund, P. C.; Kester, M.; Adair, J. H. Near-Infrared Emitting Fluorophore-Doped Calcium Phospate Nanoparticles for In Vivo Imaging of Human Breast Cancer. ACS Nano. 2008, 2 (10), 2075-2084.
4. Kester, M.; Heakal, Y.; Fox, T.; Sharma, A.; Robertson, G. P.; Morgan, T. T.; Altinoglu, E. I.; Tabakovic, A.; Parette, M. R.; Rouse, S. M.; Ruiz-Velasco, V.; Adair, J. H. Calcium phosphate nanocomposite particles for in vitro imaging and encapsulated chemotherapeutic drug delivery to cancer cells. Nano Lett. 2008, 8 (12), 4116-21.
5. Muddana, H. S.; Morgan, T. T.; Adair, J. H.; Butler, P. J. Photophysics of Cy3-Encapsulated Calcium Phosphate Nanoparticles. Nano Lett. 2009, 9 (4), 1559-1566.
6. Altinoglu, E. I.; Adair, J. H. Calcium phosphate nanocomposite particles: a safer and more effective alternative to conventional chemotherapy? Future Oncol. 2009, 5 (3), 279-81.
7. Adair, J. H.; Parette, M. P.; Altinoglu, E. I.; Kester, M. Nanoparticulate alternatives for drug delivery. ACS Nano. 2010, 4 (9), 4967-70.
8. Barth, B. M.; Sharma, R.; Altinoglu, E. I.; Morgan, T. T.; Shanmugavelandy, S. S.; Kaiser, J. M.; McGovern, C.; Matters, G. L.; Smith, J. P.; Kester, M.; Adair, J. H. Bioconjugation of calcium phosphosilicate composite nanoparticles for selective targeting of human breast and pancreatic cancers in vivo. ACS Nano. 2010, 4 (3), 1279-87.
9. Barth, B. M.; E, I. A.; Shanmugavelandy, S. S.; Kaiser, J. M.; Crespo-Gonzalez, D.; DiVittore, N. A.; McGovern, C.; Goff, T. M.; Keasey, N. R.; Adair, J. H.; Loughran, T. P., Jr.; Claxton, D. F.; Kester, M. Targeted indocyanine-green-loaded calcium phosphosilicate nanoparticles for in vivo photodynamic therapy of leukemia. ACS Nano. 2011, 5 (7), 5325-37.
10. Morgan, T. T.; Goff, T. M.; Adair, J. H. The colloidal stability of fluorescent calcium phosphosilicate nanoparticles: the effects of evaporation and redispersion on particle size distribution. Nanoscale. 2011, 3, 2044-2053.
11. Pinto, O. A.; Tabaković, A.; Goff, T. M.; Liu, Y.; Adair, J. H. Calcium Phosphate and Calcium Phosphosilicate Mediated Drug Delivery and Imaging. In Intracellular Delivery, Ferrari, M., Ed. Springer: California, 2011; Vol. 5, pp 713-744.
12. Tabakovic, A.; Kester, M.; Adair, J. H. Calcium phosphate-based composite nanoparticles in bioimaging and therapeutic delivery applications. Wiley interdisciplinary reviews. Nanomedicine and nanobiotechnology. 2012, 4 (1), 96-112.
13. Dai, J. P.; Tabakovic, A.; Loc, W.; Butler, A.; McGovern, C. O.; Adair, J. H.; Butler, J. P.; Stanley, B. A.; Smith, J. P.; Kester, M.; Matters, G. L. Development of an ultrasensitive LC-MS/MS method for determination of 5-fluorouracil in mouse plasma. Current Trends in Mass Spectrometry. 2013.
14. Barth, B. M.; Shanmugavelandy, S. S.; Kaiser, J. M.; McGovern, C.; Altinoglu, E. I.; Haakenson, J. K.; Hengst, J. A.; Gilius, E. L.; Knupp, S. A.; Fox, T. E.; Smith, J. P.; Ritty, T. M.; Adair, J. H.; Kester, M. PhotolmmunoNanoTherapy reveals an anticancer role for sphingosine kinase 2 and dihydrosphingosine-1-phosphate. ACS Nano. 2013, 7 (3), 2132-44.
15. Loc, W. S.; Smith, J. P.; Matters, G.; Kester, M.; Adair, J. H. Novel strategies for managing pancreatic cancer. World journal of gastroenterology: WIG. 2014, 20 (40), 14717-25.
16. Longley, D. B.; Harkin, D. P.; Johnston, P. G. 5-fluorouracil: mechanisms of action and clinical strategies. Nature reviews. Cancer. 2003, 3 (5), 330-8.
17. Gore, J.; Korc, M. Pancreatic cancer stroma: friend or foe? Cancer cell. 2014, 25 (6), 711-2.
18. Noordhuis, P.; Holwerda, U.; Van der Wilt, C. L.; Van Groeningen, C. J.; Smid, K.; Meijer, S.; Pinedo, H. M.; Peters, G. J. 5-Fluorouracil incorporation into RNA and DNA in relation to thymidylate synthase inhibition of human colorectal cancers. Annals of oncology: official journal of the European Society for Medical Oncology/ESMO. 2004, 15 (7), 1025-32.
19. Duschinsky, R.; Pleven, E.; Heidelberger, C. The Synthesis of 5-Fluoropyrimidines. Journal of the American Chemical Society. 1957, 79 (16), 4559-4560.
20. Diasio, R. B.; Harris, B. E. Clinical pharmacology of 5-Fluorouracil. Clin Pharmacokinet. 1989, 16 (4), 215-37.
21. van Kuilenburg, A. B.; Hausler, P.; Schalhorn, A.; Tanck, M. W.; Proost, J. H.; Terborg, C.; Behnke, D.; Schwabe, W.; Jabschinsky, K.; Maring, J. G. Evaluation of 5-fluorouracil pharmacokinetics in cancer patients with a c.1905+1G>A mutation in DPYD by means of a Bayesian limited sampling strategy. Clin Pharmacokinet. 2012, 51 (3), 163-74.
22. Kline, C. L.; El-Deiry, W. S. Personalizing colon cancer therapeutics: targeting old and new mechanisms of action. Pharmaceuticals. 2013, 6 (8), 988-1038.
23. Zhang, Y.; Kim, W. Y.; Huang, L. Systemic delivery of gemcitabine triphosphate via LCP nanoparticles for NSCLC and pancreatic cancer therapy. Biomaterials. 2013, 34 (13), 3447-58.
24. Zhang, Y.; Peng, L.; Mumper, R. J.; Huang, L. Combinational delivery of c-myc siRNA and nucleoside analogs in a single, synthetic nanocarrier for targeted cancer therapy. Biomaterials. 2013, 34 (33), 8459-68.
25. Li, J.; Yang, Y.; Huang, L. Calcium phosphate nanoparticles with an asymmetric lipid bilayer coating for siRNA delivery to the tumor. Journal of controlled release: official journal of the Controlled Release Society. 2012, 158 (1), 108-14.
26. Chen, W. H.; Lecaros, R. L.; Tseng, Y. C.; Huang, L.; Hsu, Y. C. Nanoparticle delivery of HIF1alpha siRNA combined with photodynamic therapy as a potential treatment strategy for head-and-neck cancer. Cancer letters. 2015, 359 (1), 65-74.
27. Gmeiner, W. H.; Iversen, P. L. PATENT: Oligonucleotide prodrugs containing 5-fluorouracil (FdUMP-10 polymer). 1997.
28. Gmeiner, W. H.; Skradis, A.; Pon, R. T.; Liu, J. Cytotoxicity and in-vivo tolerance of FdUMP[10]: a novel pro-drug of the TS inhibitory nucleotide FdUMP. Nucleosides & nucleotides. 1999, 18 (6-7), 1729-30.
29. Liu, J.; Kolath, J.; Anderson, J.; Kolar, C.; Lawson, T. A.; Talmadge, J.; Gmeiner, W. H. Positive interaction between 5-FU and FdUMP[10] in the inhibition of human colorectal tumor cell proliferation. Antisense Nucleic Acid Drug Dev. 1999, 9 (5), 481-6.
30. Liu, J.; Skradis, A.; Kolar, C.; Kolath, J.; Anderson, J.; Lawson, T.; Talmadge, J.; Gmeiner, W. H. Increased cytotoxicity and decreased in vivo toxicity of FdUMP[10] relative to 5-FU. Nucleosides & nucleotides. 1999, 18 (8), 1789-802.
31. Liu, J.; Kolar, C.; Lawson, T. A.; Gmeiner, W. H. Targeted Drug Delivery to Chemoresistant Cells: Folic Acid Derivatization of FdUMP[10] Enhances Cytotoxicity toward 5-FU-Resistant Human Colorectal Tumor Cells. The Journal of Organic Chemistry. 2001, 66 (17), 5655-5663.
32. Liu, C.; Willingham, M.; Liu, J.; Gmeiner, W. Efficacy and safety of FdUMP[10] in treatment of HT-29 human colon cancer xenografts. International Journal of Oncology. 2002.
33. Gmeiner, W. H.; Trump, E.; Wei, C. Enhanced DNA-directed effects of FdUMP[10] compared to 5FU. Nucleosides, nucleotides & nucleic acids. 2004, 23 (1-2), 401-10.
34. Liao, Z. Y.; Sordet, O.; Zhang, H. L.; Kohlhagen, G.; Antony, S.; Gmeiner, W. H.; Pommier, Y. A novel polypyrimidine antitumor agent HUMP[10] induces thymineless death with topoisomerase I-DNA complexes. Cancer research. 2005, 65 (11), 4844-51.
35. Bijnsdorp, I. V.; Comijn, E. M.; Padron, J. M.; Gmeiner, W. H.; Peters, G. J. Mechanisms of action of FdUMP[10]: metabolite activation and thymidylate synthase inhibition. Oncology reports. 2007, 18 (1), 287-91.
36. Gmeiner, W. H.; Reinhold, W. C.; Pommier, Y. Genome-Wide mRNA and microRNA Profiling of the NCI 60 Cell Line Screen and Comparison of HUMP[10] with fluorouracil, floxuridine, and Top1 Poisons. 2010.
37. Pardee, T. S.; Gomes, E.; Jennings-Gee, J.; Caudell, D.; Gmeiner, W. H. Unique dual targeting of thymidylate synthase and topoisomerasel by FdUMP[10] results in high efficacy against AML and low toxicity. Blood. 2012, 119 (15), 3561-70.
38. Gmeiner, W. H.; Pardee, T. S. PATENT: Method of treating acute myelogenous leukemia (FdUMP-10 polymer). 2013.
39. Gmeiner, W. H.; Lema-Tome, C.; Gibo, D.; Jennings-Gee, J.; Milligan, C.; Debinski, W. Selective anti-tumor activity of the novel fluoropyrimidine polymer F10 towards G48a orthotopic GBM tumors. Journal of neuro-oncology. 2014, 116 (3), 447-54.
40. Pardee, T. S.; Stadelman, K.; Jennings-Gee, J.; Caudell, D. L.; Gmeiner, W. H. The poison oligonucleotide F10 is highly effective against acute lymphoblastic leukemia while sparing normal hematopoietic cells. Oncotarget. 2014, 5 (12), 4170-9.
41. Schmitz, J. C.; Chu, E.; Gmeiner, W. H. PATENT: Modified siRNA Molecules Incorporating 5-Fluoro-2′-Deoxyuridine Residues to Enhance Cytotoxicity. 2014.
42. Gmeiner, W. H.; Boyacioglu, O.; Stuart, C. H.; Jennings-Gee, J.; Balaji, K. C. The cytotoxic and pro-apoptotic activities of the novel fluoropyrimidine F10 towards prostate cancer cells are enhanced by Zn(2+)-chelation and inhibiting the serine protease Omi/HtrA2. The Prostate. 2015, 75 (4), 360-9.
43. Kotchetkov, R.; Groschel, B.; Gmeiner, W. H.; Krivtchik, A. A.; Trump, E.; Bitoova, M.; Cinatl, J.; Kornhuber, B.; Cinatl, J. Antineoplastic activity of a novel multimeric gemcitabine-monophosphate prodrug against thyroid cancer cells in vitro. Anticancer research. 2000, 20 (5A), 2915-22.
44. Nanninga, L. B. Formation Constants and Hydrolysis at 100 C of Calcium and Magnesium Complexes of Adenosinetri-, di-, and monophosphate. J. Phys. Chem. 1957, 61, 1144-1149.
45. Burton, K. Formation constants for the complexes of adenosine di- or tri-phosphate with magnesium or calcium ions. The Biochemical journal. 1959, 71 (2), 388-95.
46. Smith, R. M.; Alberty, R. A. The Apparent Stability Constants of Ionic Complexes of Various Adenosine Phosphates with Monovalent Cations. J Phys Chem-Us. 1956, 60 (2), 180-184.
47. DiStefano, V.; Neuman, W. F. Calcium Complexes of Adenosinetriphosphate and adenosinediphosphate and their significance in calcification in vitro. J. Biol. Chem. 1953, 200, 759-763.
48. Cabral, H.; Matsumoto, Y.; Mizuno, K.; Chen, Q.; Murakami, M.; Kimura, M.; Terada, Y.; Kano, M. R.; Miyazono, K.; Uesaka, M.; Nishiyama, N.; Kataoka, K. Accumulation of sub-100 nm polymeric micelles in poorly permeable tumours depends on size. Nature nanotechnology. 2011, 6 (12), 815-23.
49. Stober, W.; Fink, A.; Bohn, E. Controlled Growth of Monodisperse Silica Spheres in the Micron Size Range. J. Colloid Interfac. Sci. 1968, 26, 62-69.
50. Bogush, G. H.; Tracy, M. A.; Zukoski, C. F. Preparation of Monodisperse Silica Particles—Control of Size and Mass Fraction. J Non-Cryst Solids. 1988, 104 (1), 95-106.
51. Bogush, G. H.; Zukoski, C. F. Uniform Silica Particle-Precipitation—an Aggregative Growth-Model. J Colloid Interf Sci. 1991, 142 (1), 19-34.
52. Sheehan, J.; Cruickshank, P.; Boshart, G. Notes—A Convenient Synthesis of Water-Soluble Carbodiimides. The Journal of Organic Chemistry. 1961, 26 (7), 2525-2528.
53. Staros, J. V. N-hydroxysulfosuccinimide active esters: bis(N-hydroxysulfosuccinimide) esters of two dicarboxylic acids are hydrophilic, membrane-impermeant, protein cross-linkers. Biochemistry. 1982, 21 (17), 3950-5.
54. Arumugam, T.; Ramachandran, V.; Fournier, K. F.; Wang, H.; Marquis, L.; Abbruzzese, J. L.; Gallick, G. E.; Logsdon, C. D.; McConkey, D. J.; Choi, W. Epithelial to mesenchymal transition contributes to drug resistance in pancreatic cancer. Cancer Res. 2009, 69 (14), 5820-8.
55. Tsujie, M.; Nakamori, S.; Nakahira, S.; Takahashi, Y.; Hayashi, N.; Okami, J.; Nagano, H.; Dono, K.; Umeshita, K.; Sakon, M.; Monden, M. Human equilibrative nucleoside transporter 1, as a predictor of 5-fluorouracil resistance in human pancreatic cancer. Anticancer research. 2007, 27 (4B), 2241-9.
56. Kurata, N.; Fujita, H.; Ohuchida, K.; Mizumoto, K.; Mahawithitwong, P.; Sakai, H.; Onimaru, M.; Manabe, T.; Ohtsuka, T.; Tanaka, M. Predicting the chemosensitivity of pancreatic cancer cells by quantifying the expression levels of genes associated with the metabolism of gemcitabine and 5-fluorouracil. Int J Oncol. 2011, 39 (2), 473-82.
57. Commisso, C.; Davidson, S. M.; Soydaner-Azeloglu, R. G.; Parker, S. J.; Kamphorst, J. J.; Hackett, S.; Grabocka, E.; Nofal, M.; Drebin, J. A.; Thompson, C. B.; Rabinowitz, J. D.; Metallo, C. M.; Vander Heiden, M. G.; Bar-Sagi, D. Macropinocytosis of protein is an amino acid supply route in Ras-transformed cells. Nature. 2013, 497 (7451), 633-7.
58. Harmsen, S.; Huang, R.; Wall, M. A.; Karabeber, H.; Samii, J. M.; Spaliviero, M.; White, J. R.; Monette, S.; O'Connor, R.; Pitter, K. L.; Sastra, S. A.; Saborowski, M.; Holland, E. C.; Singer, S.; Olive, K. P.; Lowe, S. W.; Blasberg, R. G.; Kircher, M. F. Surface-enhanced resonance Raman scattering nanostars for high-precision cancer imaging. Science translational medicine. 2015, 7 (271), 271ra7.
59. van der Wilt, C. L.; Backus, H. H.; Smid, K.; Comijn, L.; Veerman, G.; Wouters, D.; Voorn, D. A.; Priest, D. G.; Bunni, M. A.; Mitchell, F.; Jackman, A. L.; Jansen, G.; Peters, G. J. Modulation of both endogenous folates and thymidine enhance the therapeutic efficacy of thymidylate synthase inhibitors. Cancer research. 2001, 61 (9), 3675-81.
60. Patel, K.; Yerram, S. R.; Azad, N. A.; Kern, S. E. A thymidylate synthase ternary complex-specific antibody, FTS, permits functional monitoring of fluoropyrimidines dosing. Oncotarget. 2012, 3 (7), 678-85.
61. Matuo, R.; Sousa, F. G.; Escargueil, A. E.; Grivicich, I.; Garcia-Santos, D.; Chies, J. A.; Saffi, J.; Larsen, A. K.; Henriques, J. A. 5-Fluorouracil and its active metabolite FdUMP cause DNA damage in human SW620 colon adenocarcinoma cell line. Journal of applied toxicology: JAT. 2009, 29 (4), 308-16.
in vivoin vitroin vivoin vivoin vivoin vitro
62. de Sousa Cavalcante L, Monteiro G. Gemcitabine: Metabolism and molecular mechanisms of action, sensitivity and chemoresistance in pancreatic cancer. European journal of pharmacology. 2014; 741:8-16.
63. Ewald B, Sampath D, Plunkett W. Nucleoside analogs: molecular mechanisms signaling cell death. Oncogene. 2008; 27(50):6522-37.
64. Baker J A R, Wickremsinhe E R, Li C H, Oluyedun O A, Dantzig A H, Hall S D, et al. Pharmacogenomics of Gemcitabine Metabolism: Functional Analysis of Genetic Variants in Cytidine Deaminase and Deoxycytidine Kinase. Drug Metabolism and Disposition. 2013; 41(3): 541-5.
65. Heinemann V, Xu Y Z, Chubb S, Sen A, Hertel L W, Grindey G B, et al. Cellular elimination of 2′,2′-difluorodeoxycytidine 5′-triphosphate: a mechanism of self-potentiation. Cancer research. 1992; 52(3): 533-9.
66. Abbruzzese J L, Grunewald R, Weeks E A, Gravel D, Adams T, Nowak B, et al. A phase I clinical, plasma, and cellular pharmacology study of gemcitabine. Journal of clinical oncology: official journal of the American Society of Clinical Oncology. 1991; 9(3):491-8.
67. Wang W B, Yang Y, Zhao Y P, Zhang T P, Liao Q, Shu H. Recent studies of 5-fluorouracil resistance in pancreatic cancer. World journal of gastroenterology: WJG. 2014; 20(42): 15682-90.
68. Lockshin A, Mondal K, Danenberg P V. Spectroscopic studies of ternary complexes of thymidylate synthetase, deoxyribonucleotides, and folate analogs. The Journal of biological chemistry. 1984; 259(18): 11346-52.
69. Clawson G A, Abraham T, Pan W, Tang X, Linton S S, McGovern C O, et al. A Cholecystokinin B Receptor-Specific DNA Aptamer for Targeting Pancreatic Ductal Adenocarcinoma. Nucleic acid therapeutics. 2016.

What is claimed is:

1. A method of treating a cancer cell or imaging targeted tissue in a subject, the method comprising,
a) contacting at least one cancer cell or targeted tissue with an effective amount of a drug or imaging agent encapsulated in a calcium phosphosilicate nanoparticle (CPNSP).
b) exposing said at least one cancer cell or said targeted tissue to said CPNSP encapsulated drug or targeting agent to obtain a desired result.

2. The method of claim 1, wherein said drug comprises a 5-fluorouracil (5FU) metabolite or a gemcitabine metabolite.

3. The method of claim 1, wherein said drug comprises 5-fluoro-2′-deoxyuridine 5′-monophosphate (FdUMP).

4. The method of claim 1, wherein said drug comprises gemcitabine monophosphate (dFdCMP).

5. The method of claim 1, wherein said nanoparticle comprises on its surface polyethylene glycol (PEG).

6. The method of claim 1 wherein said at least one cancer cell comprises at least one pancreatic cancer cell, said drug comprises 5-fluoro-2′-deoxyuridine 5′-monophosphate (FdUMP) and proliferation of said at least one pancreatic cancer cell is reduced compared to said cancer cells not contacted with said CPNSP.

7. The method of claim 1, wherein said cancer cell comprises at least one pancreatic cancer cell, said drug comprises 5-fluoro-2′-deoxyuridine 5′-monophosphate (FdUMP) and proliferation of said at least one pancreatic cancer cell is reduced compared to cancer cells contact with non-encapsulated FdUMP.

8. The method of claim 7 wherein said FdUMP encapsulated in said CPNSP reduced proliferation of said cancer cells at least as much as that reduced by non-encapsulated FdUMP.

9. The method of claim 8, wherein the dose of said FdUMP encapsulated in CPNSP is 1000 time less than the dose of said non-encapsulated FdUMP.

10. The method of claim 1, wherein said drug comprises 5-fluoro-2′-deoxyuridine 5′-monophosphate (FdUMP) and said desired response is inhibition of thymidylate synthase.

11. The method of claim 1, wherein said cancer cell comprises at least one pancreatic cancer cell, said drug comprises FdUMP and said desired response comprises arrest of said at least one cancer cell prior to G1 phase.

12. The method of claim 1, wherein said at least one cancer cell comprises a PANC-1 tumor.

13. The method of claim 1, wherein said CPSNP further comprises on the surface of said CPSNP a CCKBR aptamer.

14. A method of producing encapsulated drugs or imaging agents, the method comprising, encapsulating said drug or imaging agent into a calcium phosphosilicate nanoparticle.

15. The method of claim 14, wherein said nanoparticles are produced that are 10 nm up to 200 nm in diameter.

16. The method of claim 14, wherein the amount of said drug or imaging agent are encapsulated is at least 10% up to 100%.

17. The method of claim 14, wherein the nanoparticle surface is conjugated with a polyethylene glycol (PEG) chosen from a PEG with terminal groups selected from hydroxyl, methoxy, malemidie, amine, carboxy functional groups.

18. The method of claim 14, wherein said drug is a 5-fluorouracil (5-FU) metabolite or a gemcitabine metabolite.

19. A composition comprising a nanoparticle, said composition comprising a drug or imaging agent nano-encapsulated with calcium phosphosilicate nanoparticle (CPNSP), said drug or imaging agent having increased efficacy for a desired outcome compared to efficacy where said drug or imaging agent is not encapsulated with CPNSP.

20. The composition of claim 19, said drug comprising a 5-fluorouracil (5-FU) metabolite or gemcitabine metabolite, said nanoparticle surface conjugated with methoxy PEG, said drug in localized within said CPSNP and said drug has increased efficacy in arrest or reducing proliferation of pancreatic cancer cells.
